(19) United States
(12) Patent Application Publication
(10) Pub. No.: US 2016/0289175 A1
(43) Pub. Date: Oct. 6, 2016

(54) CREATINE ANALOGS AND THE USE THEREOF

(71) Applicant: ULTRAGENYX PHARMACEUTICAL INC., Novato, CA (US)

(72) Inventors: Steven Jungles, Novato, CA (US); Yiumo Chan, Novato, CA (US); Emil Kakkis, Novato, CA (US)

(21) Appl. No.: 15/034,658

(22) PCT Filed: Nov. 5, 2014
(86) PCT No.: PCT/US14/64028

§ 371 Date: May 5, 2016

Related U.S. Application Data
(60) Provisional application No. 61/899,975, filed on Nov. 5, 2013.

(51) Int. Cl. C07C 279/22

(52) U.S. Cl. CPC C07C 279/22

(57) ABSTRACT

The present invention provides novel creatine analogs useful for treating any creatine deficiency disorders and methods of treating and preventing creatine deficiencies utilizing the present compounds and the pharmaceutical compositions or formulations thereof. Certain embodiments seek to increase the lipophilicty of novel creatine analogs with the goal of improving their bioavailability.

### CROSS-REFERENCE TO RELATED APPLICATION

This application claims the benefit of priority to U.S. Provisional Patent Application No. 61/899,975 filed Nov. 5, 2013, which is herein incorporated by reference in its entirety for all purposes.

### FIELD OF THE INVENTION

The present invention relates to creatine analogs useful for treating syndromes and illnesses associated with creatine deficiency.

### BACKGROUND OF THE INVENTION

As a naturally occurring amino acid, creatine is produced in human body and also found in meat and fish. Creatine is predominately used as a fuel source in muscle. Specifically, creatine helps to supply energy to cells in the body by increasing the formation of adenosine triphosphate (ATP). In a cell's mitochondria, creatine interacts reversibly with adenosine triphosphate (ATP), which is caused by the action of the creatine kinase enzyme with a formation of creatine phosphate and adenosine diphosphate (ADP). This interaction maintains of the ATP concentration at a constant level at the moments of its intense consumption. Approximately 95% of the human body's total creatine is located in skeletal muscle.

It is known that dysfunction in energy metabolism can cause many diseases. Particularly, the loss of cellular ATP due to oxygen and glucose deprivation during ischemia is a cause of tissue death. Creatine phosphate represents a reserve of macroergic phosphate in maintaining the membrane potential, activation of metabolites or contractive activity of a cell. It maintains the ATP level along with an increasing of energy consumption in a cell, i.e. restores an ortho-phosphate residue on ADP. Creatine phosphate and Creatine are also allosteric regulators of cell processes. The creatine kinase system is a key biochemical mechanism that prevents ATP depletion in mammalian cells. The level of creatine phosphate in a cell is an important predictor of resistance to ischemic insult, and remaining stores of creatine phosphate are correlated with the extent of tissue damage. Thus, creatine can be used for treating cardiac and brain ischemia, neuronal degeneration, organ transplant viability, and muscle fatigue and other diseases related to creatine deficiency. Nowadays, the treatment of creatine biosynthesis defects has yielded significant clinical improvement. However, the use of creatine and creatine phosphate is limited because of poor solubility and instability in aqueous media at physiological pH-values. Moreover, creatine is poorly absorbed from the gastrointestinal tract. This requires high usage doses of creatine. For the effective use of creatine, compositions produced at the present time require consumption in an amount up to 20 g per day. Such high doses of creatine may lead to negative consequences for the organism, such as disturbance of nitrogen exchange, gastrointestinal disorders, diarrhea, etc. Some clinical studies based on the use of creatine supplemented by amino acids such as L-arginine and L-glycine showed no improvement of clinical features in long follow-up of patients. Thus, successful therapeutic strategies still need to be discovered in order to treat the creatine transporter defect.

### SUMMARY OF THE INVENTION

The present invention provides novel creatine analogs useful for treating any creatine deficiency disorders and methods of treating and preventing creatine deficiencies utilizing the present compounds and the pharmaceutical compositions or formulations thereof.

In one embodiment, the present invention provides a compound having structural Formula (I):

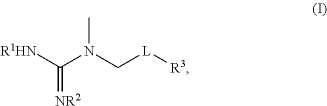

or a pharmaceutically acceptable salt or solvate thereof; wherein:

R¹ is hydrogen, —C(O)—NH—R⁴, —C(O)—O—R⁴, an amino acid residue, a dipeptide residue, or a tripeptide residue;

R² is hydrogen, —C(O)—NH—R⁵, —C(O)—O—R⁵, an amino acid residue, a dipeptide residue, or a tripeptide residue;

L is —C(O)—O— or —C(O)—NH—;

R³ is hydrogen, alkyl, alkenyl, C(O)—R⁶, an amino acid residue, a dipeptide residue, a tripeptide residue, a glucose residue, a phospholipid moiety, or a triglyceride moiety; or alternatively R¹ and R³, taken together with the atoms to which they are attached, form a heterocyclic ring; and

R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl.

In one embodiment, Formula (I) has the following provisos:

R¹, R² and R³ are not all hydrogen, but at least one of R¹, R² and R³ is hydrogen;

when R¹ and R² are hydrogen and L is —C(O)—NH—; then Formula (I) does not include a compound selected from the group consisting of Creatinyl-γ-Aminobutyric Acid Ethyl Ester, Creatinyl-L-Phenylalanine Amide, Creatinyl-L-Phenylalanine Amide, Creatinyl-Glycine Benzyl Ester, Creatinyl-Tyrosine Amide, Creatinyl-Glycine Ethylamide, Creatinyl-Phenylalanyl-Arginyl-Glycine Ethyl Ester, and Creatinyl-Phenylalanine; and

when R¹ and R² are hydrogen and L is —C(O)—O—; then R³ is not alkyl or C(O)—R⁶.

In another embodiment, the present invention provides a pharmaceutical composition comprising a compound of the present invention, or a pharmaceutically acceptable salt or solvate thereof; and a pharmaceutically acceptable excipient.

In another embodiment, the present invention provides a method for treating creatine deficiency in a patient in need thereof comprising administering to the patient a therapeutically effective amount of a compound of the present invention, or a pharmaceutically acceptable salt or solvate thereof.

### DETAILED DESCRIPTIONS OF THE INVENTION

Various embodiments and advantages of the present invention will be set forth in part in the description that follows, and in part will be obvious from the description, or may be learned by practice of the invention. It is to be understood that both the foregoing general description and the following detailed description are exemplary and explanatory only and are not restrictive of the invention as described.

### DEFINITIONS

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (i.e., meaning “including, but not limited to”). The endpoints of all ranges directed to the same component or property are inclusive and independently combinable.

Reference to “about” a value or parameter herein includes (and describes) variations that are directed to that value or parameter per se. For example, description referring to “about X” includes description of “X”.

The term “present compound(s)” or “compound(s) of the present invention” refers to compounds encompassed by structural formulae disclosed herein and includes any subgenus and specific compounds within these formulae whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule. The term “tautomer” as used herein refers to isomers that change into one another with great ease so that they can exist together in equilibrium.

“Alkyl,” by itself or as part of another substituent, refers to a saturated branched, straight-chain or cyclic monovalent hydrocarbon radical derived by the removal of one hydrogen atom from a single carbon atom of a parent alkane. The term “alkyl” includes “cycloakyl” as defined herein below. Typical alkyl groups include, but are not limited to, methyl; ethyl; propyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl). C₁-C₆ alkyl is also known as “lower alkyl”.

It is noted that when an alkyl group is further connected to another atom, it becomes an “alkylene” group. In other words, the term “alkylene” refers to a divalent alkyl. For example, —CH₂CH₃ is an ethyl, while —CH₂CH₂— is an ethylene. That is, “Alkylene,” by itself or as part of another substituent, refers to a saturated or unsaturated, branched, straight-chain or cyclic divalent hydrocarbon radical derived by the removal of two hydrogen atoms from a single carbon atom or two different carbon atoms of a parent alkane, alkene or alkyne. The term “alkylene” includes “cycloalkylene” as defined herein below. The term “alkylene” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. In some embodiments, an alkylene group comprises from 1 to 20 carbon atoms (C₁-C₂₀ alkylene). In other embodiments, an alkylene group comprises from 1 to 10 carbon atoms (C₁-C₁₀ alkylene). In still other embodiments, an alkylene group comprises from 1 to 6 carbon atoms (C₁-C₆ alkylene).

“Alkenyl,” by itself or as part of another substituent, refers to an unsaturated branched, straight-chain or cyclic monovalent hydrocarbon radical having at least one carbon-carbon double bond derived by the removal of one hydrogen atom from a single carbon atom of a parent alkene. The term “alkenyl” includes “cycloalkenyl” as defined herein below. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic monovalent hydrocarbon radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy,” by itself or as part of another substituent, refers to a radical of the formula —O—R¹⁹⁹, where R¹⁹⁹ is alkyl or substituted alkyl as defined herein.

“Acyl” by itself or as part of another substituent refers to a radical —C(O)R²⁰⁰, where R²⁰⁰ is hydrogen, alkyl, substituted alkyl, aryl, substituted aryl, arylalkyl, substituted arylalkyl, heteroalkyl, substituted heteroalkyl, heteroarylalkyl or substituted heteroarylalkyl as defined herein. Representative examples include, but are not limited to formyl, acetyl, cyclohexylcarbonyl, cyclohexylmethylcarbonyl, benzoyl, benzylcarbonyl and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. That is, arylakyl can also be considered as an alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Carbocyclic,” or “Carbocyclyl,” by itself or as part of another substituent, refers to a saturated or partially saturated, buy not aromatic, cyclic monovalent hydrocarbon radical, including cycloalkyl, cycloalkenyl, and cycloalkynyl as defined herein. Typical carbocyclyl groups include, but are not limited to, groups derived from cyclopropane, cyclobutane, cyclopentane, cyclohexane, and the like. In some embodiments, the cycloalkyl group comprises from 3 to 10 ring atoms (C₃-C₁₀ cycloalkyl). In other embodiments, the cycloalkyl group comprises from 3 to 7 ring atoms (C₃-C₇ cycloalkyl). The carbocyclyl may be further substituted by one or more heteroatoms including, but not limited to, N, P, O, S, and Si, which attach to the carbon atoms of the cycloalkyl via monovalent or multivalent bond.

“Heteroalkyl,” by themselves or as part of other substituents, refer to alkyl groups, in which one or more of the carbon atoms, are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl group. Typical heteroatomic groups which can be included in these groups include, but are not limited to, —O—, —S—, —O—O—, —S—S—, —O—S—, —NR²⁰¹R²⁰²—, ═N—N═, —N═N—, —N═N—NR²⁰³R²⁰⁴, —PR²⁰⁵—, —P(O)₂—, —POR²⁰⁶—, —O—P(O)₂—, —SO—, —SO₂—, —SnR²⁰⁷R²⁰⁸— and the like, where R²⁰¹, R²⁰², R²⁰³, R²⁰⁴, R²⁰⁵, R²⁰⁶, R²⁰⁷ and R²⁰⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heterocyclic,” or “Heterocyclyl,” by itself or as part of another substituent, refers to a carbocyclic radical in which one or more carbon atoms are independently replaced with the same or different heteroatom. The heterocyclyl may be further substituted by one or more heteroatoms including, but not limited to, N, P, O, S, and Si, which attach to the carbon atoms of the heterocyclyl via monovalent or multivalent bond. Typical heteroatoms to replace the carbon atom(s) include, but are not limited to, N, P, O, S, Si, etc. Typical heterocyclyl groups include, but are not limited to, groups derived from epoxides, azirines, thiiranes, imidazolidine, morpholine, piperazine, piperidine, pyrazolidine, pyrrolidone, quinuclidine, and the like. In some embodiments, the heterocyclyl group comprises from 3 to 10 ring atoms (3-10 membered heterocyclyl) In other embodiments, the heterocyclyl group comprise from 5 to 7 ring atoms (5-7 membered heterocyclyl). A cycloheteroalkyl group may be substituted at a heteroatom, for example, a nitrogen atom, with a (C₁-C₆) alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of“heterocyclyl.” A heterocyclyl group may be attached to the remainder of the molecule via a ring carbon atom or a ring heteroatom. As used herein, hererocyclyl includes a glucose residue, a nucleoside residue, and a ascorbic acid residue.

“Halo,” by itself or as part of another substituent refers to a radical —F, —Cl, —Br or —I.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

An “amide” refers to an organic compound that contains the functional group consisting of a carbonyl group linked to a nitrogen atom. For example, an amide group can be represented by the following structural formula:

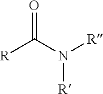

R is an optionally substituted hydrocarbon moiety;
R′ and R″ are independently hydrogen or optionally substituted hydrocarbon moiety.

A “lactam” group is a cyclic amide. That is, a lactam is an amide with the above structural formula where R and R′ or R and R″, taken together with the carbon and nitrogen atoms to which they are attached, form an optionally substituted cyclic group.

An “ester” refers to an organic compound derived by reacting/condensing an oxoacid with a hydroxyl compound. For example, an amide group can be represented by the following structural formula:

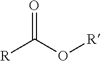

R and R′ are independently hydrogen or optionally substituted hydrocarbon moiety.

A “lactone” group is a cyclic ester. That is, a lactone is an ester with the above structural formula where R and R′, taken together with the carbon and oxygen atoms to which they are attached, form an optionally substituted cyclic group which can be saturated, unsaturated, or aromatic.

A “urea” or “carbamide” refers to an organic compound having the following structural formula:

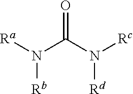

R^{a}, R^{b}, R^{c}, and R^{d} are independently hydrogen or optionally substituted hydrocarbon moiety.

A cyclic urea is a urea with the above structural formula where any two of R^{a}, R^{b}, R^{c}, and R^{d}, taken together with the carbon and nitrogen atoms to which they are attached, form an optionally substituted cyclic group which can be saturated, unsaturated, or aromatic.

A “carbonate” refers to an organic compound having the following structural formula:

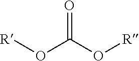

R′ and R″ are independently hydrogen or optionally substituted hydrocarbon moiety.

A cyclic carbonate is a carbonate with the above structural formula where R′ and R″, taken together with the carbon and oxygen atoms to which they are attached, form an optionally substituted cyclic group which can be saturated, unsaturated, or aromatic.

A “carbamate” refers to an organic compound having the following structural formula:

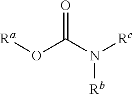

R^{a}, R^{b}, and R^{c} are independently hydrogen or optionally substituted hydrocarbon moiety.

A cyclic carbamate is a carbamate with the above structural formula where any two of R^{a} and R^{b}, or R^{a} and R^{c}, taken together with the carbon and nitrogen/oxygen atoms to which they are attached, form an optionally substituted cyclic group which can be saturated, unsaturated, or aromatic.

“Hydrocarbon” refers to an organic compound consisting of hydrogen and carbon. Hydrocarbons can be straight, branched, or cyclic; and include arenes, alkanes, alkenes, cycloalkanes, alkynes, and etc. The term “substituted hydrocarbon” refers to a hydrocarbon where a carbon or hydrogen atom is replaced by an atom which is not carbon or hydrogen. The substituted hydrocarbons include substituted arenes, substituted alkanes, heteroalkanes, substituted alkenes, heteroalkenes, substituted cycloalkanes, heterocycloalkanes, substituted alkynes, and etc.

“Prodrug” refers to an inactive derivative of a therapeutically active agent that will be converted to the active agent in vivo. That is, a prodrug is a precursor of a drug.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl, and trityl ethers as well as alkyl ethers, tetrahydropyranyl ethers, trialkylsilyl ethers and allyl ethers.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Solvate” means a compound formed by solvation (the combination of solvent molecules with molecules or ions of the solute), or an aggregate that consists of a solute ion or molecule, i.e., a compound of the present invention, with one or more solvent molecules. When water is the solvent, the corresponding solvate is “hydrate”.

By “pharmaceutically acceptable” is meant a material that is not biologically or otherwise undesirable, i.e., the material may be incorporated into a pharmaceutical composition administered to a patient without causing any significant undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

“N-oxide”, also known as amine oxide or amine-N-oxide, means a compound that derives from a compound of the present invention via oxidation of an amine group of the compound of the present invention. An N-oxide typically contains the functional group R₃N⁺—O⁻ (sometimes written as R₃N═O or R₃N→O).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and cycloheteroalkyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR)NR^{c}R^{c}, where R^{a}, R^{b} and R^{c} are as previously defined.

Substituent groups from the above lists useful for substituting other specified groups or atoms will be apparent to those of skill in the art.

The term “substituted” specifically envisions and allows for one or more substitutions that are common in the art. However, it is generally understood by those skilled in the art that the substituents should be selected so as to not adversely affect the useful characteristics of the compound or adversely interfere with its function. Suitable substituents may include, for example, halogen groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, arylalkyl or heteroarylalkyl groups, arylalkoxy or heteroarylalkoxy groups, amino groups, alkyl- and dialkylamino groups, carbamoyl groups, alkylcarbonyl groups, carboxyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, cycloalkyl groups, cyano groups, C₁-C₆ alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof. In the case of substituted combinations, such as “substituted arylalkyl,” either the aryl or the alkyl group may be substituted, or both the aryl and the alkyl groups may be substituted with one or more substituents. Additionally, in some cases, suitable substituents may combine to form one or more rings as known to those of skill in the art.

The term “optionally substituted” denotes the presence or absence of the substituent group. For example, optionally substituted alkyl includes both unsubstituted alkyl and substituted alkyl. The substituents used to substitute a specified group can be further substituted, typically with one or more of the same or different groups selected from the various groups specified above.

“Carrier” refers to a diluent, adjuvant, excipient or vehicle with which a compound is administered.

The term “Amino acid” refers to an organic compounds that contains an amino group (NH₂), a carboxyl group (COOH), and any of various side groups. For example, the twenty two amino acids that are naturally incorporated into polypeptides (a.k.a. natural amino acids or naturally occurring amino acids) have the structural formula NH₂CHRCOOH, wherein R is a moiety including hydrogen, optionally substituted hydrocarbon moiety, etc. It is commonly known that certain amino acids have two stereoisomers designated as L and D amino acids. Amino acids as mentioned herein include L isomer, D isomer, or a mixture thereof. Furthermore, any of the L, D, or mixed amino acids may further contain additional stereoisomeric center(s) in their structures. The amino and carboxyl groups may be located at alpha, beta, gamma, delta, or other positions. Amino acids suitable for the present invention can be naturally occurring amino acid or non-naturally occurring (e.g., synthetic) amino acid. Examples of the amino acids include, but are not limited to, alanine, arginine, asparagine, aspartic acid, cysteine, glutamic acid, gluamine, glycine, histidine, isoleucine, leucine, lysine, methionine, phenylalanine, proline, serine, threonine, tryptophan, tyrsosine, valine, selenocysteine, pyrolysine, and any derivatives thereof.

The term “peptidyl group”, as used herein, denotes an organic moiety derived from one or more amino acid(s) by removal of a hydrogen atom from the NH₂ and/or OH group of the amino acid(s). When the peptidyl group is derived from a single amino acid, it is a monopeptidyl group. When the peptidyl group is derived from a molecule of multiple amino acids, it is a multipeptidyl group, e.g., dipeptidyl or tripeptidyl. The amino acids in a multipeptidyl group is linked with each other via amide bond(s). The term “dipeptide”, as used herein, denotes a molecule containing two amino acids joined by a single amide bond, while the term “tripeptide”, as used herein, denotes a molecule containing three amino acids joined by two amide bonds.

By “immediate release” or “instant release”, it is meant a conventional or non-modified release in which greater than or equal to about 75% of the active agent is released within two hours of administration, specifically within one hour of administration.

By “sustained release”, it is meant a dosage form in which the release of the active agent is controlled or modified over a period of time. Sustained can mean, for example, extended-, controlled-, delayed-, timed-, or pulsed-release at a particular time. Alternatively, controlled can mean that the release of the active agent is extended for longer than it would be in an immediate-release dosage form, e.g., at least over several hours.

By “effective amount” or “therapeutically effective amount” it is meant the amount of the present compound that, when administered to a patient for treating a disease, such as one related to Creatine deficiency, is sufficient to effect such treatment for the disease. The “effective amount” or “therapeutically effective amount” will vary depending on the active agent, the disease and its severity, and the age, weight, and other conditions of the patient to be treated.

The terms “treating” and “treatment”, as used herein, refer to an approach for obtaining beneficial or desired results including clinical results. For purposes of this invention, beneficial or desired clinical results include, but are not limited to, one or more of the following: decreasing the severity and/or frequency one or more symptoms resulting from the disease, diminishing the extent of the disease, stabilizing the disease (e.g., preventing or delaying the worsening of the disease), delay or slowing the progression of the disease, ameliorating the disease state, increasing production of Creatine, the sialylation precursor CMP-Creatine (e.g., increasing intracellular production of Creatine) and restoring the level of sialylation in muscle and other proteins, decreasing the dose of one or more other medications required to treat the disease, and/or increasing the quality of life. “Treating” a patient with a compound or composition described herein includes management of an individual to inhibit or cause regression of a disease or condition.

“Prophylaxis” or “prophylactic treatment” “or preventive treatment” refers to prevention of the occurrence of symptoms and/or their underlying cause, for example, prevention of a disease or condition in a patient susceptible to developing a disease or condition (e.g., at a higher risk, as a result of genetic predisposition, environmental factors, predisposing diseases or disorders, or the like). Prophylaxis includes HIBM myopathy in which chronic disease changes in the muscles are irreversible and for which animal model data suggests treatment benefit in prophylaxis.

The term “patient” refers to an animal, for example, a mammal and includes, but is not limited to, human, bovine, horse, feline, canine, rodent, or primate. Preferably, the patient is a human.

### Embodiments of the Compound

In one aspect, the present invention is directed to creatine analogs which are converted, at least in part, to creatine upon administration to a patient.

In one embodiment, the present invention is directed to a compound represented by a structural Formula (I):

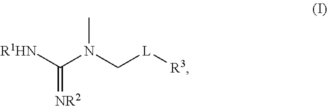

or a pharmaceutically acceptable salt or solvate thereof; wherein, R¹ is hydrogen, —C(O)—NH—R⁴, —C(O)—O—R⁴, an amino acid residue, a dipeptide residue, or a tripeptide residue; R² is hydrogen, —C(O)—NH—R⁵, —C(O)—O—R⁵, an amino acid residue, a dipeptide residue, or a tripeptide residue; L is —C(O)—O— or —C(O)—NH—; R³ is hydrogen, alkyl, alkenyl, C(O)—R⁶, an amino acid residue, a dipeptide residue, a tripeptide residue, a glucose residue, a phospholipid moiety, or a triglyceride moiety; or alternatively R¹ and R³, taken together with the atoms to which they are attached, form a heterocyclic ring; and R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl.

In one embodiment of Formula (I), R¹, R² and R³ are not all hydrogen, but at least one of R¹, R² and R³ is hydrogen.

In one embodiment of Formula (I), when R¹ and R² are hydrogen and L is —C(O)—NH—; then Formula (I) does not include a compound selected from the group consisting of Creatinyl-γ-Aminobutyric Acid Ethyl Ester, Creatinyl-L-Phenylalanine Amide, Creatinyl-L-Phenylalanine Amide, Creatinyl-Glycine Benzyl Ester, Creatinyl-Tyrosine Amide, Creatinyl-Glycine Ethylamide, Creatinyl-Phenylalanyl-Arginyl-Glycine Ethyl Ester, and Creatinyl-Phenylalanine. In another embodiment of Formula (I), R¹ and R² are hydrogen and L is —C(O)—NH—; then Formula (I) does not include a compound wherein R is a residue of a naturally occurring amino acid.

In one embodiment of Formula (I), when R¹ and R² are hydrogen and L is —C(O)—O—; then R³ is not alkyl or C(O)—R⁶.

In one embodiment of Formula (I), when R³ is not hydrogen, then at least one of R¹ and R² is hydrogen.

In one embodiment of the present invention, the compound of Formula (I) demonstrates increased hydrophobicity or increased uptake by a carrier-mediated transporter as compared to the uptake of creatine, wherein the carrier-mediated transporter is selected from the group consisting of amino acid transporter, monocarboxylic acid transporter, small peptide transporter, glucose transporter, glutathione transporter, ascorbic acid transporter, and nucleoside transporter.

In one embodiment of Formula (I), the compound is represented by Formula (II):

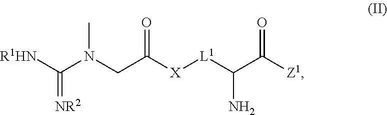

wherein, R¹ is hydrogen, —C(O)—NH—R⁴, —C(O)—O—R⁴, an amino acid residue, a dipeptide residue, or a tripeptide residue; R² is hydrogen, —C(O)—NH—R⁵, —C(O)—O—R⁵, an amino acid residue, a dipeptide residue, or a tripeptide residue; X is O or NH; L¹ is alkylene, substituted alkylene, arylene, substituted arylene; aralkylene, or substituted aralkylene; Z¹ is C(O)—R⁶, OH, OR⁷, an amino acid residue, a dipeptide residue, a tripeptide residue, a glucose residue, a phospholipid moiety, or a triglyceride moiety; R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and R⁷ is alkyl.

In one embodiment of Formula (II), R¹ and R² are both hydrogen.

In one embodiment of Formula (II), X is O or NH; and Z¹ is an amino acid residue.

In one embodiment of Formula (II), X is O or NH; and Z¹ is a dipeptide residue or a tripeptide residue.

In one embodiment of Formula (II), R¹ is —C(O)—NH—R⁴, or —C(O)—O—R⁴; R² is hydrogen; R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; X is O or NH; L¹ is alkylene, substituted alkylene, arylene, substituted arylene; aralkylene, or substituted aralkylene; and Z¹ is OH.

In one embodiment of Formula (II), R¹ is hydrogen; R² is —C(O)—NH—R⁵, or —C(O)—O—R⁵; R⁵ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; X is O or NH; L¹ is alkylene, substituted alkylene, arylene, substituted arylene; aralkylene, or substituted aralkylene; and Z¹ is OH.

In one embodiment of Formula (II), R¹ is a dipeptide residue, or a tripeptide residue; R² is hydrogen; R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; X is O or NH; L¹ is alkylene, substituted alkylene, arylene, substituted arylene; aralkylene, or substituted aralkylene; and Z¹ is OH.

In one embodiment of Formula (II), one of R¹ and R² is not hydrogen; Z¹ is OR⁷ or C(O)—R⁶; R⁶ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and R⁷ is short-, medium, or long-chain alkyl.

In one embodiment of Formula (I), the compound is represented by Formula (III):

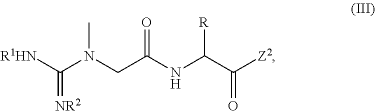

wherein, R¹ is hydrogen, —C(O)—NH—R⁴, —C(O)—O—R⁴, an amino acid residue, a dipeptide residue, or a tripeptide residue; R² is hydrogen, —C(O)—NH—R⁵, —C(O)—O—R⁵, an amino acid residue, a dipeptide residue, or a tripeptide residue; Z² is OH, OR⁷, C(O)—R⁶, an amino acid residue, a dipeptide residue, a tripeptide residue, a glucose residue, a phospholipid moiety, or a triglyceride moiety; and R, R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl.

In one embodiment of Formula (III), R¹ and R² are both hydrogen.

In one embodiment of Formula (III), Z² is an amino acid residue.

In one embodiment of Formula (III), Z² is a dipeptide residue or a tripeptide residue.

In one embodiment of Formula (III), R¹ is —C(O)—NH—R⁴, or —C(O)—O—R⁴; R² is hydrogen; R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and Z¹ is OH.

In one embodiment of Formula (III), R¹ is hydrogen; R² is —C(O)—NH—R⁵, or —C(O)—O—R⁵; R⁵ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and Z¹ is OH.

In one embodiment of Formula (III), R¹ is a dipeptide residue, or a tripeptide residue; R² is hydrogen; and Z¹ is OH.

In one embodiment of Formula (III), one of R¹ and R² is not hydrogen; Z² is OR⁷ or C(O)—R⁶; R⁶ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and R⁷ is short-, medium, or long-chain alkyl.

In one embodiment of Formula (I), the compound is represented by Formula (II), wherein R¹ is —C(O)—NH—R⁴ or —C(O)—O—R⁴; R² is hydrogen; and R⁴ and Z¹, taken together with the atoms to which they are attached, form a heterocyclic ring.

In one embodiment of Formula (I), the compound is represented by Formula (III), wherein R¹ is —C(O)—NH—R⁴ or —C(O)—O—R⁴; R² is hydrogen; and R⁴ and Z², taken together with the atoms to which they are attached, form a heterocyclic ring.

In one embodiment of Formula (I), R¹ and R² are both hydrogen; L is —C(O)—O—; and R³ is a glucose residue.

In one embodiment of Formula (I), R¹ is —C(O)—NH—R⁴ or —C(O)—O—R⁴; R² is hydrogen; L is —C(O)—O—; R³ is hydrogen; and R⁴ is heterocyclyl or substituted heterocyclyl. In another embodiment, R⁴ is a glucose residue, a nucleoside residue, or a ascorbic acid residue.

In one embodiment of Formula (I), R¹ and R² are both hydrogen; L is —C(O)—O—; and R³ is a phospholipid moiety.

In one embodiment of Formula (I), R¹ and R² are both hydrogen; L is —C(O)—O—; and R³ is a triglyceride moiety. In one embodiment of Formula (I), the triglyceride moiety of R³ contains at least one odd-numbered carbon (e.g., C3, C5, C7, C9, C11, C13, or C15) fatty acid moiety, such as propanoate, pentanoate, heptanoate, and nonanoate. In one embodiment of Formula (I), the triglyceride moiety of R³ contains two odd-numbered carbon fatty acid moieties which can be the same or different. In another embodiment, the triglyceride moiety of R³ contains one odd-numbered carbon (e.g., C3, C5, C7, C9, C11, C13, or C15) fatty acid moiety and another functional group, such as a phospholipid moiety. In one embodiment, the odd-numbered carbon fatty acid moiety is heptanoate. In one embodiment, the compound of Formula (I) is represented by structural Formula (Ia):

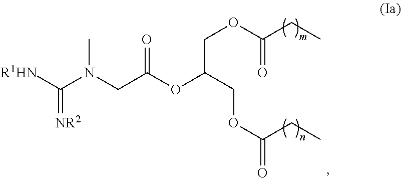

wherein R¹ and R² are defined the same as Formula (I) above; and m and n are independently 1, 3, 5, 7, 9, or 11.

In one embodiment of Formula (Ia), R¹ is hydrogen. In another embodiment of Formula (Ia), R² is hydrogen. In yet another embodiment of Formula (Ia), R¹ and R² are both hydrogen. In one embodiment of Formula (Ia), R¹ and R² are both hydrogen; m and n are both 5. This odd-numbered chain fatty acid moiety may produce beneficial effects on mitochondrial energy metabolism. Specifically, the oxidation of acetyl-CoA by the citric acid cycle (CAC) and subsequent oxidative phosphorylation by the electron transport chain produces the most ATP in aerobic metabolism. The CAC intermediates α-ketoglutarate and oxaloacetate are precursors for the neurotransmitters glutamate, GABA and aspartate. Increased neurotransmission could reduce the levels of CAC intermediates and subsequently acetyl-CoA oxidation and energy production. The odd-numbered chain fatty acids can provide anaplerotic propionyl-CoA molecules without overloading the system with nitrogen, sodium or acid.

In some specific embodiments, the compounds of the present invention are selected from the group consisting of

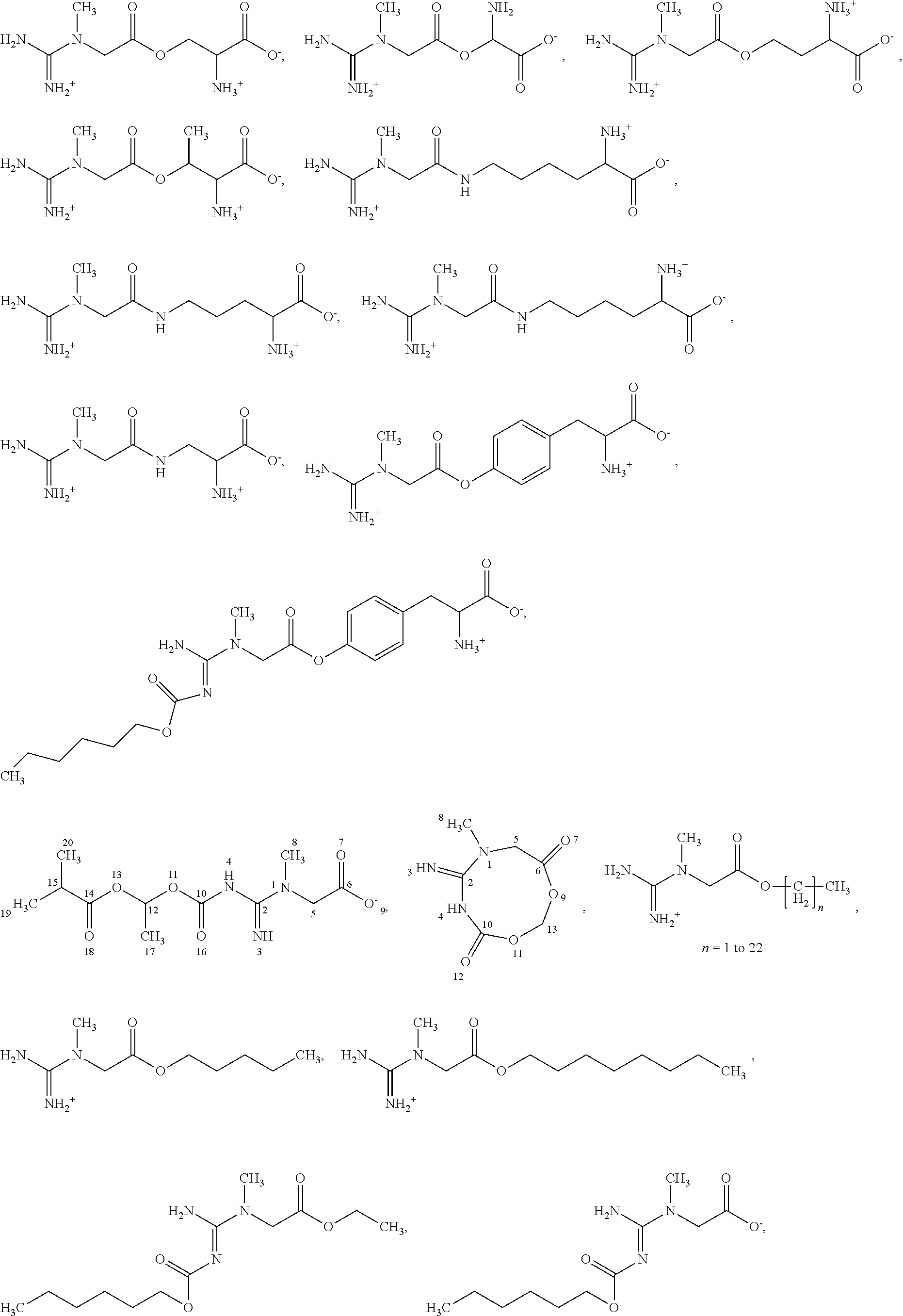

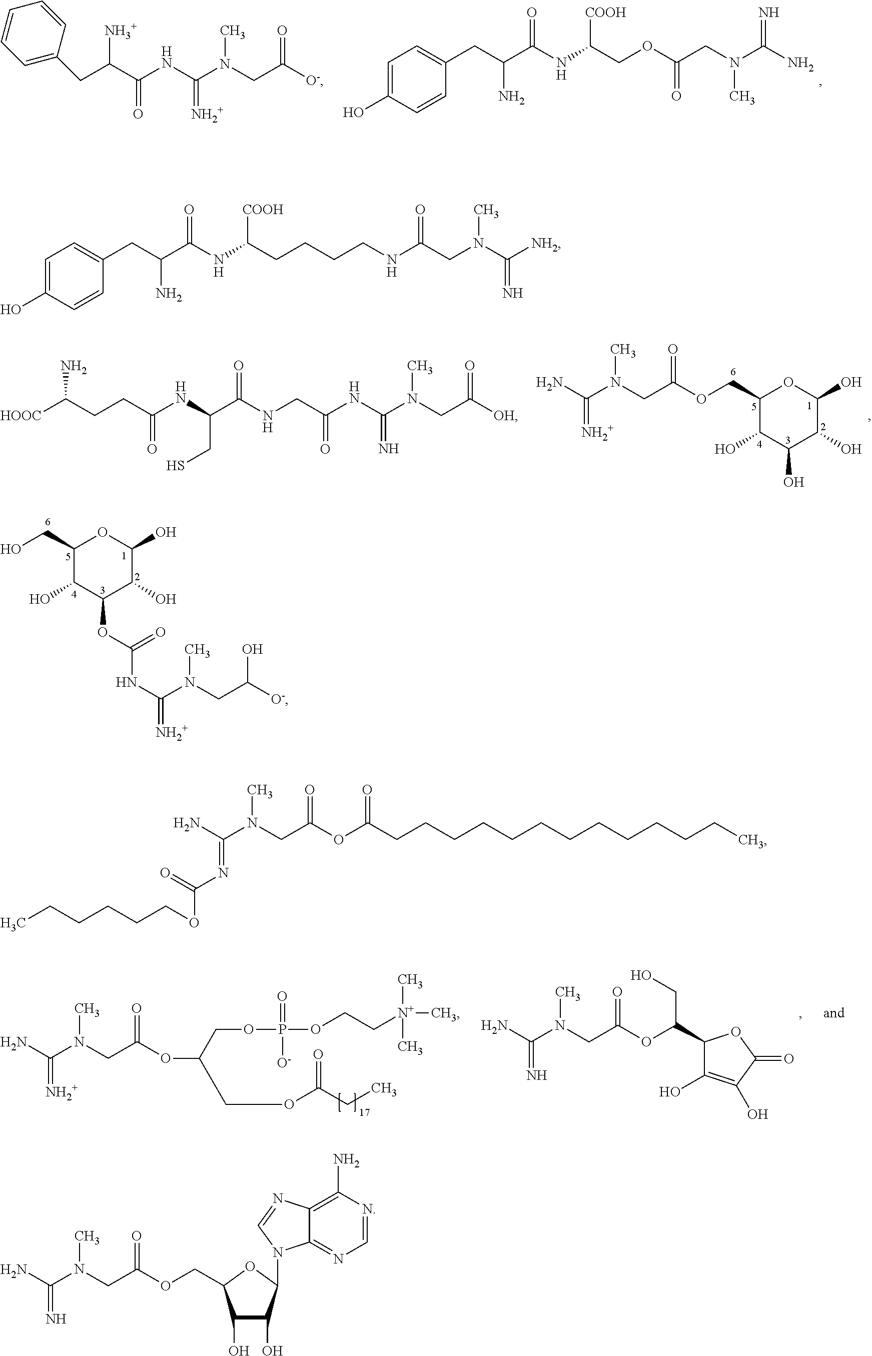

### Embodiments of the Utilities of the Present Compounds

In one embodiment of the present invention, the present compounds can be used for the treatment of creatine deficiencies by administering an effective amount of the present compound, or a pharmaceutically acceptable salt or solvate thereof, to a patient in need of such treatment. In another embodiment, the method comprises administering a present compound, or a pharmaceutically acceptable salt or solvate thereof, to a patient in need of such treatment; wherein upon administration, the compound, or a pharmaceutically acceptable salt or solvate thereof, continuously provides a therapeutically effective amount of creatine for more than about 4 hours. In some embodiments, the diseases, disorders, or conditions associated with creatine deficiency is ischemia, ischemic Reperfusion Injury, transplant Perfusion, neurodegenerative Diseases, Parkinson's Disease, Alzheimer's Disease, Huntington's Disease, Amyotrophic Lateral Sclerosis, Amyotrophic lateral sclerosis (ALS), creatine transporter dysfunction including cerebral creatine deficiency syndromes (CCDS), Multiple Sclerosis, psychotic disorders, Schizophrenia, bipolar disorder, anxiety, epilepsy including myoclonic epilepsy, and seizure including seizures with clinical manifestations in muscle, muscular dystrophy, myopathy associated with mitochondrial diseases, such as mitochondrial myopathy, genetic diseases affecting the creatine kinase system, muscle fatigue, muscle strength, organ and cell viability, or diseases related to glucose level regulation. As used herein, “muscular dystrophy” refers to muscle diseases that are typically characterized by progressive skeletal muscle weakness, defects in muscle proteins, and the death of muscle cells and tissue. Muscular dystrophy often weakens the musculoskeletal system and hampers locomotion. Examples of muscular dystrophies include, but are not limited to, Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, myotonic muscular dystrophy, oculopharyngeal muscular dystrophy, or any combinations thereof. More details can be found in patent publication, U.S. Pat. No. 8,202,852, the contents of which are incorporated by reference.

In other embodiments, the method can continuously provide a therapeutically effective amount of creatine for a period from about 1 hour to about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours.

In one embodiment, the therapeutically effective amount refers to the amount administered to the patient. In yet another embodiment, the therapeutically effective amount refers to the amount delivered to muscle tissue of the individual. The present compounds, upon administration, are converted to creatine in vivo. That is, the present compounds, upon administration, are metabolized to one or more compounds in the creatine pathway or derivatives thereof (including creatine itself).

The term “creatine transporter dysfunction” includes a disorder characterized by an inborn error creatine synthesis or of the creatine transporter or other aberrant creatine transport function in the brain. The aberrant creatine transport function in the brain may cause the subject to suffer from a low concentration of creatine in the brain of a subject due to creatine transporter dysfunction. In this disorder, impaired energy metabolism is believed to be associated with impaired learning dysfunction, cognitive function, and neurological syndrome, such as developmental delay, mild epilepsy and severe expressive language impairment. For example, creatine transporter dysfunction can lead to cerebral creatine deficiency syndromes (CCDS) which include a group of inborn errors of creatine biosynthesis and transport through the cellular membranes. These diseases are associated with severe neurologic features: mental retardation, expressive speech and language delay, pervasive developmental disorder, autism, autism spectrum disorder, autistic-like behavior, asperger syndrome, attention deficit hyperactivity disorder (ADHD), epilepsy including myoclonic epilepsy, and seizure including seizures with clinical manifestations in muscle. They are characterized by a lack of creatine in the brain and metabolic disturbances in the nervous system since the creatine is involved in the cellular phosphocreatine energy system. The only way to treat patients is to restore the cerebral creatine pool by bringing creatine into the brain. The absence of functional creatine transporters at the blood-brain barrier (BBB) may prevent the entry of creatine into the brain, thus affecting the cognitive functions. For instance, creatine amino acids and phosphocreatine-Mg complex show neuroprotective activity in in vivo animal models of cerebral stroke, ischemia or hypoxia. In addition, a 9-week treatment with cyclocreatine as treatment in SLC6A8 knockout mice resulted in an increase in phosphocreatine and phosphocyclocreatine 31P-MRS signals as well as normalization of behavioral test findings.

As the brain cells are the ultimate target for creatine delivery, it is imperative that it has to cross the blood brain barrier (BBB). Creatine does not cross the BBB efficiently by itself. In some embodiments, the compounds of the present invention can pass the BBB and/or be release inside the targeted cells as free creatine.

In some embodiments, the present compounds are stable in biological fluids, to enter cells by either passive diffusion or active transport, and to release the corresponding creatine analog into the cellular cytoplasm. Such prodrug analogs can also cross important barrier tissues such as the intestinal mucosa, the blood-brain barrier, and the blood-placental barrier. Because of the ability to pass through biological membranes, these prodrugs can restore and maintain energy homeostasis in ATP depleted cells via the creatine kinase system, and rapidly restore ATP levels to protect tissues from further ischemic stress.

Compounds of the present invention and the present compositions can be useful in treating of diseases, disorders, or conditions in a patient associated with a dysfunction in energy metabolism. In certain embodiments, a disease associated with a dysfunction in energy metabolism is selected from ischemia, oxidative stress, a neurodegenerative disease, ischemic reperfusion injury, a cardiovascular disease, multiple sclerosis, a psychotic disease, and muscle fatigue. In certain embodiments, treating a disease comprises effecting energy homeostasis in a tissue or organ affected by the disease.

### Ischemia

The present compounds can be used to treat acute or chronic ischemic diseases, disorders, or conditions. Ischemia is an imbalance of oxygen supply and demand in a cell, tissue, or organ. Ischemia is characterized by hypoxia, including anoxia, insufficiency of metabolic substrates for normal cellular bioenergetics, and accumulation of metabolic waste. The present compounds can be used to treat acute or chronic ischemia. In certain embodiments, a compound or composition can be particularly useful in acute or emergency treatment of ischemia in tissue or organs characterized by high energy demand such as the brain, neurons, heart, lung, kidney, or the intestine.

The neuron is limited by its availability of energy-generating substrates, being limited to using primarily glucose, ketone bodies, or lactate. The neuron does not produce or store glucose or ketone bodies and cannot survive for any significant period of time without a substrate, which is absorbed and used directly or indirectly from the bloodstream. Thus, a constant supply of an energy-generating substrate must be present in the blood at all times in an amount sufficient to supply the entire brain and the rest of the body with energy-generating substrates.

Lack of oxygen or glucose prevents or limits the ability of neurons to synthesize ATP. The intracellular creatine/phosphocreatine system can to some extent compensate for the lack of oxygen or glucose. Creatine kinase catalyses the synthesis of phosphocreatine from creatine in normal brain tissue. Under conditions of ATP depletion, phosphocreatine can donate its phosphate group to ADP to resynthesize ATP. However, neuronal phosphocreatine content is limited following complete anoxia or ischemia phosphocreatine is also rapidly depleted. ATP depletion is believed to block Na⁺/K⁺ ATPases causing neurons to depolarize and lose membrane potential.

Neuroprotective effects of compounds of the present invention can be determined using animal models of cerebral ischemia such as those described, for example, in Cimino et al., Neuroloxicol 2005, 26(5), 9929-33; Konstas et al., Neurocril Care 2006, 4(2). 168-78; Wasterlain et al., Neurology 1993, 43(11), 2303-10; and Zhu et al., J Neuroscience 2004, 24(26), 5909-5912.

In certain embodiments, the present compounds can be used to treat a cardiovascular disease, including cerebral ischemia (stroke) and myocardial ischemia (heart infarction).

Cardiovascular disease includes hypertension, heart failure such as congestive heat failure or heart failure following myocardial infarction, arrhythmia, diastolic dysfunction such as left ventricular diastolic dysfunction, diastolic heart failure, or impaired diastolic filling, systolic dysfunction, ischemia such as myocardial ischemia, cardiomyopathy such as hypertrophic cardiomyopathy and dilated cardiomyopathy, sudden cardiac death, myocardial fibrosis, vascular fibrosis, impaired arterial compliance, myocardial necrotic lesions, vascular damage in the heart, vascular inflammation in the heart, myocardial infarction including both acute post-myocardial infarction and chronic post-myocardial infarction conditions, coronary angioplasty, left ventricular hypertrophy, decreased ejection fraction, coronary thrombosis, cardiac lesions, vascular wall hypertrophy in the heart, endothelial thickening, myocarditis, and coronary artery disease such as fibrinoid necrosis or coronary arteries. Ventricular hypertrophy due to systemic hypertension in association with coronary ischemic heart disease is recognized as a major risk factor for sudden death, post infarction heart failure, and cardiac rupture. Patients with severe left ventricular hypertrophy are particularly susceptible to hypoxia or ischemia.

### Ischemic Reperfusion Injury

In certain embodiments, the present compounds provided by the present disclosure can be used to treat a condition associated with ischemic reperfusion injury or reduce ischemic reperfusion injury. Ischemic reperfusion injury can be associated with oxygen deprivation, neutrophil activation, and/or myeloperoxidase production. Ischemic reperfusion injury can be the result of a number of disease states or can be iatrogenically induced, for example, by blood clots, stenosis, or surgery.

In certain embodiments, the present compounds can be used to treat stroke, a fatal or non-fatal myocardial infarction, peripheral vascular disease, tissue necrosis, and kidney failure, and post-surgical loss of muscle tone resulting from ischemic reperfusion injury. In certain embodiments, the methods and compositions provided by the present disclosure reduce or mitigate the extent of ischemic reperfusion injury.

In certain embodiments, the present compounds can be used to treat, reduce or prevent ischemic reperfusion injury associated with occlusion or blood diversion due to vessel stenosis, thrombosis, accidental vessel injury, or surgical procedures.

In certain embodiments, compounds of the present invention and compositions thereof can also be used to treat any other condition associated with ischemic reperfusion such as myocardial infarction, stroke, intermittent claudication, peripheral arterial disease, acute coronary syndrome, cardiovascular disease and muscle damage as a result of occlusion of a blood vessel.

In certain embodiments, the present compounds can be used to treat reperfusion injury associated with myocardial infarction, stenosis, at least one blood clot, stroke, intermittent claudication, peripheral arterial disease, acute coronary syndrome, cardiovascular disease, or muscle damage as a result of occlusion of a blood vessel.

In certain embodiments, the present compounds can be used in conjunction with cardiac surgery, for example, in or with cardioplegic solutions to prevent or minimize ischemia or reperfusion injury to the myocardium. In certain embodiments, the methods and compositions can be used with a cardiopulmonary bypass machine during cardiac surgery to prevent or reduce ischemic reperfusion injury to the myocardium.

In certain embodiments, the methods and compositions provided by the present disclosure can protect muscle and organs such as, for example, the heart, liver, kidney, brain, lung, spleen and steroidogenic organs, e.g. thyroid, adrenal glands, and gonads, from damage as a result of ischemia reperfusion injury.

The present compounds can be used to treat ischemic reperfusion injury in a tissue or organ by contacting the tissue or organ with an effective amount of the compound or pharmaceutical composition. The tissue or organ may be in a patient or outside of a patient, i.e., extracorporeal. The tissue or organ can be a transplant tissue or organ, and the compound or pharmaceutical composition can be contacted with the transplant tissue or organ before removal, during transit, during transplantation, and/or after the tissue or organ is transplanted in the recipient.

In certain embodiments, compounds or the present compositions can be used to treat ischemic perfusion injury caused by surgery, such as cardiac surgery. A compound or pharmaceutical composition can be administered before, during, and/or after surgery. In certain embodiments, a compound or pharmaceutical composition provided by the present disclosure can be used to treat ischemic reperfusion injury to muscle, including cardiac muscle, skeletal muscle, or smooth muscle, and in certain embodiments, to treat ischemic reperfusion injury to an organ such as the heart, lung, kidney, spleen, liver, neuron, or brain. A compound of the present invention or pharmaceutical composition thereof can be administered before, during, and/or after surgery.

In certain embodiments, compounds of the present invention or the present compositions can be used to treat ischemic perfusion injury to a muscle, including cardiac muscle, skeletal muscle, and smooth muscle.

The efficacy of a compound of the present invention for treating ischemic reperfusion injury may be assessed using animal models and in clinical trials. Examples of useful methods for assessing efficacy in treating ischemic reperfusion injury are disclosed, for example, in Prass et al., J Cereh Blood Flow Metab 2007, 27(3), 452-459; Arya et al., Life Sci 2006, 79(1), 38-44; Lee et al., Eur. J. Pharmacol 2005, 523(1-3), 101-108; and Bisgaier et al., U.S. Application Publication No. 2004/0038891. Useful methods for evaluating transplant perfusion/reperfusion are described, for example, in Ross et al., Am J. Physiol—Lung Cellular Mol. Physiol. 2000, 279(3), L528-536.

### Transplant Perfusion

In certain embodiments, compounds of the present invention or pharmaceutical compositions thereof can be used to increase the viability of organ transplants by perfusing the organs with a compound of the present invention or pharmaceutical compositions thereof. Increased creatine phosphate levels are expected to prevent or minimize ischemic damage to an organ. In certain embodiments, the present compounds can be used to treat, prevent or reduce ischemia reperfusion injury in extracorporeal tissue or organs.

### Neurodegenerative Diseases

Neurodegenerative diseases featuring cell death can be categorized as acute, e.g., stroke, traumatic brain injury, spinal cord injury, and chronic, e.g., amyotrophic lateral sclerosis, Huntington's disease, Parkinson's disease, and Alzheimer's disease. Although these diseases have different causes and affect different neuronal populations, they share similar impairment in intracellular energy metabolism.

Acute and chronic neurodegenerative diseases are illnesses associated with high morbidity and mortality and few options are available for their treatment. A characteristic of many neurodegenerative diseases, which include stroke, brain trauma, spinal cord injury, amyotrophic lateral sclerosis, Huntington's disease, Alzheimer's disease, and Parkinson's disease, is neuronal-cell death. Cell death occurs by necrosis or apoptosis. Necrotic cell death in the central nervous system follows acute ischemia or traumatic injury to the brain or spinal cord. It occurs in areas that are most severely affected by abrupt biochemical collapse, which leads to the generation of free radicals and excitotoxins. Mitochondrial and nuclear swelling, dissolution of organelles, and condensation of chromatin around the nucleus are followed by the rupture of nuclear and cytoplasmic membranes and the degradation of DNA by random enzymatic cuts. Apoptotic cell death can be a feature of both acute and chronic neurological diseases. Apoptosis occurs in areas that are not severely affected by an injury. For example, after ischemia, there is necrotic cell death in the core of the lesion, where hypoxia is most severe, and apoptosis occurs in the penumbra, where collateral blood flow reduces the degree of hypoxia. Apoptotic cell death is also a component of the lesion that appears after brain or spinal cord injury. In chronic neurodegenerative diseases, apoptosis is the predominant form of cell death. In apoptosis, a biochemical cascade activates proteases that destroy molecules required for cell survival and others that mediate a program of cell death. Caspases directly and indirectly contribute to the morphologic changes of the cell during apoptosis (Friedlander, N Engl J Med 2003, 348(14), 1365-75). Oral creatine supplementation has been shown to inhibit mitochondrial cytochrome C release and downstream caspase-3 activation, and ATP depletion inhibition of the caspase-mediated cell death cascades in cerebral ischemia (Zhu et al., J Neurosci 2004, 24(26), 5909-5912) indicating that manipulation of the creatine kinase system may be effective in controlling apoptotic cell death in chronic neurodegenerative diseases.

Creatine administration shows neuroprotective effects, particularly in animal models of Parkinson's disease, Huntington's disease, and ALS (Wyss and Schulze, Neuroscience 2002, 112(2), 243-260, which is incorporated by reference herein in its entirety) and it is recognized that the level of oxidative stress may be a determinant of metabolic determination in a variety of neurodegenerative diseases.

The efficacy of administering a compound of the present invention for treating Parkinson's disease may be assessed using animal and human models of Parkinson's disease and clinical studies. Animal and human models of Parkinson's disease are known (see, e.g., O'Neil et al., CNS Drug Rev. 2005, 11(1), 77-96; Faulkner et al., Ann. Pharmacother. 2003, 37(2), 282-6; Olson et al., Am. J. Med. 1997, 102(1), 60-6; Van Blercom et al., Clin Neuropharmacol. 2004, 27(3), 124-8; Cho et al., Biochem. Biophys. Res. Commun. 2006, 341, 6-12; Emborg, J. Neuro. Meth. 2004, 139, 121-143; Tolwani et al., Lab Anim Sci 1999, 49(4), 363-71; Hirsch et al., J Neural Transm Suppl 2003, 65, 89-100; Orth and Tabrizi, Mov Disord 2003, 18(7), 729-37; Betarbet et al., Bioessays 2002, 24(4), 308-18; and McGeer and McGeer, Neurobiol Aging 2007, 28(5), 639-647).

The efficacy of administering a compound of the present invention for treating Alzheimer's disease may be assessed using animal and human models of Alzheimer's disease and clinical studies. Useful animal models for assessing the efficacy of compounds for treating Alzheimer's disease are disclosed, for example, in Van Dam and De Dyn, Nature Revs Drug Disc 2006, 5, 956-970; Simpkins et al., Ann NY Acad Sci. 2005, 1052, 233-242; Higgins and Jacobsen, Behav Pharmacol 2003, 14(5-6), 419-38; Janus and Westaway, Physiol Behav 2001, 73(5), 873-86; and Conn, ed., “Handbook of Models in Human Aging,” 2006, Elsevier Science & Technology.

The efficacy of administering a compound of the present invention for treating Huntington's disease may be assessed using animal and human models of Huntington's disease and clinical studies. Animal models of Huntington's disease are disclosed, for example, in Riess and Hoersten, U.S. Application Publication No. 2007/0044162; Rubinsztein, Trends in Genetics. 2002, 18(4), 202-209; Matthews et al., J. Neuroscience 1998, 18(1), 156-63; Tadros et al., Pharmacol Biochem Behav 2005, 82(3), 574-82, and in Kaddurah-Daouk et al., U.S. Pat. No. 6,706,764, and U.S. Application Publication Nos. 2002/0161049, 2004/0106680, and 2007/0044162. A placebo-controlled clinical trial evaluating the efficacy of creatine supplementation to treat Huntington's disease is disclosed in Verbessem et al., Neurology 2003, 61, 925-230.

The efficacy of administering a compound of the present invention for treating ALS may be assessed using animal and human models of ALS and clinical studies. Natural disease models of ALS include mouse models (motor neuron degeneration, progressive motor neuropathy, and wobbler) and the hereditary canine spinal muscular atrophy canine model (Pioro and Mitsumoto, Clin Neurosci. 19954996, 3(6), 375-85). Experimentally produced and genetically engineered animal models of ALS can also useful in assessing therapeutic efficacy (see e.g., Doble and Kennelu, Amyotroph Lateral Scler Other Motor Neuron Disord. 2000, 1(5), 301-12; Grieb, Folia Neuropathol. 2004, 42(4), 239-48; Price et al., Rev Neurol (Paris), 1997, 153(8-9), 484-95; and Klivenyi et al., Nat Med 1999, 5, 347-50). Specifically, the SOD 1-G93A mouse model is a recognized model for ALS. Examples of clinical trial protocols useful in assessing treatment of ALS are described, for example, in Mitsumoto, Amyotroph Lateral Scler Other Motor Neuron Disord. 2001, 2 Suppl 1, S10-S14; Meininger, Neurodegener Dis 2005, 2, 208-14; and Ludolph and Sperfeld, Neurodegener Dis. 2005, 2(3-4), 215-9.

### Multiple Sclerosis

Multiple sclerosis (MS) is a multifaceted inflammatory autoimmune disease of the central nervous system caused by an autoimmune attack against the isolating axonal myelin sheets of the central nervous system. Demyelination leads to the breakdown of conduction and to severe disease with destruction of local axons and irreversible neuronal cell death. The symptoms of MS are highly varied with each individual patient exhibiting a particular pattern of motor, sensible, and sensory disturbances. MS is typified pathologically by multiple inflammatory foci, plaques of demyelination, gliosis, and axonal pathology within the brain and spinal cord, all of which contribute to the clinical manifestations of neurological disability (see e.g., Wingerchuk, Lab Invest 2001, 81, 263-281; and Virley, NeruoRx 2005, 2(4), 638-649). Although the causal events that precipitate the disease are not fully understood, most evidence implicates an autoimmune etiology together with environmental factors, as well as specific genetic predispositions. Functional impairment, disability, and handicap are expressed as paralysis, sensory and octintive disturbances spasticity, tremor, a lack of coordination, and visual impairment, which impact on the quality of life of the individual. The clinical course of MS can vary from individual to individual, but invariably the disease can be categorized in three forms: relapsing-remitting, secondary progressive, and primary progressive. Several studies implicate dysfunction of creatine phosphate metabolism with the etiology and symptoms of the disease (Minderhoud et al., Arch Neurol 1992, 49(2), 161-5; He et al., Radiology 2005, 234(1), 211-7; Tartaglia et al., Arch Neurology 2004, 61(2), 201-207; Duong et al., J Neurol 2007 Apr. 20; and Ju et al., Magnetic Res Imaging 2004, 22, 427-429), although creatine ingestion alone does not appear to be effective in improving exercise capacity in individuals with MS (Lambert et al., Arch Phys Med Rehab 2003, 84(8), 1206-1210).

Assessment of MS treatment efficacy in clinical trials can be accomplished using tools such as the Expanded Disability Status Scale (Kurtzke, Neurology 1983, 33, 1444-1452) and the MS Functional Composite (Fischer et al., Mult Scler, 1999, 5, 244-250) as well as magnetic resonance imaging lesion load, biomarkers, and self-reported quality of life (see e.g., Kapoor, Cur Opinion Neurol 2006, 19, 255-259). Animal models of MS shown to be useful to identify and validate potential therapeutics include experimental autoimmune/allergic encephalomyelitis (EAE) rodent models that simulate the clinical and pathological manifestations of MS (Werkerle and Kurschus, Drug Discovery Today: Disease Models, Nervous System Disorders. 2006, 3(4), 359-367; Gijbels et al., Neurosci Res Commun 2000, 26, 193-206; and Hofstetter et al., J Immunol 2002, 169, 117-125), and nonhuman primate EAE models ('t Hart et al., Immunol Today 2000, 21, 290-297).

### Psychotic Disorders

In certain embodiments, compounds of the present invention or pharmaceutical compositions thereof can be used to treat psychotic disorders such as, for example, schizophrenia, bipolar disorder, and anxiety.

### Schizophrenia

Schizophrenia is a chronic, severe, and disabling brain disorder that affects about one percent of people worldwide, including 3.2 million Americans. Schizophrenia encompasses a group of neuropsychiatric disorders characterized by dysfunctions of the thinking process, such as delusions, hallucinations, and extensive withdrawal of the patient's interests from other people. Schizophrenia includes the subtypes of paranoid schizophrenia characterized by a preoccupation with delusions or auditory hallucinations, hebephrenic or disorganized schizophrenia characterized by disorganized speech, disorganized behavior, and flat or inappropriate emotions; catatonic schizophrenia dominated by physical symptoms such as immobility, excessive motor activity, or the assumption of bizarre postures; undifferentiated schizophrenia characterized by a combination of symptoms characteristic of the other subtypes; and residual schizophrenia in which a person is not currently suffering from positive symptoms but manifests negative and/or cognitive symptoms of schizophrenia (see DSM-IV-TR classifications 295.30 (Paranoid Type), 295.10 (Disorganized Type), 295.20 (Catatonic Type), 295.90 (Undifferentiated Type), and 295.60 (Residual Type); Diagnostic and Statistical Manual of Mental Disorders, 4^{th} Edition, American Psychiatric Association, 297-319, 2005). Schizophrenia includes these and other closely associated psychotic disorders such as schizophreniform disorder, schizoaffective disorder, delusional disorder, brief psychotic disorder, shared psychotic disorder, psychotic disorder due to a general medical condition, substance-induced psychotic disorder, and unspecified psychotic disorders (DSM-IV-TR, 4^{th} Edition, pp. 297-344, American Psychiatric Association, 2005).

The efficacy of the compound of the present invention and pharmaceutical compositions thereof for treating schizophrenia may be determined by methods known to those skilled in the art. For example, negative, positive, and/or cognitive symptom(s) of schizophrenia may be measured before and after treatment of the patient. Reduction in such symptom(s) indicates that a patient's condition has improved. Improvement in the symptoms of schizophrenia may be assessed using, for example, the Scale for Assessment of Negative Symptoms (SANS), Positive and Negative Symptoms Scale (PANSS) (see, e.g., Andreasen, 1983, Scales for the Assessment of Negative Symptoms (SANS), Iowa City, Iowa; and Kay et al., Schizophrenia Bulletin 1987, 13, 261-276), and using Cognitive Deficits tests such as the Wisconsin Card Sorting Test (WCST) and other measures of cognitive function (see, e.g., Keshavan et al., Schizophr Res 2004, 70(2-3), 187-194; Rush, Handbook of Psychiatric Measures, American Psychiatric Publishing 2000; Sajatovic and Ramirez, Rating Scales in Mental Health, 2nd ed, Lexi-Comp, 2003, Keefe, et al., Schizophr Res. 2004, 68(2-3), 283-97; and Keefe et al., Neuropsychopharmacology. 19 Apr. 2006.

The efficacy of the compound of the present invention and pharmaceutical compositions thereof may be evaluated using animal models of schizophrenic disorders (see e.g., Geyer and Moghaddam, in “Neuropsychopharmacology,” Davis et al., Ed., Chapter 50, 689-701, American College of Neuropsychopharmacology, 2002). For example, conditioned avoidance response behavior (CAR) and catalepsy tests in rats are shown to be useful in predicting antipsychotic activity and EPS effect liability, respectively (Wadenberg et al., Neuropsychopharmacology, 2001, 25, 633-641).

### Bipolar Disorder

Bipolar disorder is a psychiatric condition characterized by periods of extreme mood. The moods can occur on a spectrum ranging from depression (e.g., persistent feelings of sadness, anxiety, guilt, anger, isolation, and/or hopelessness, disturbances in sleep and appetite, fatigue and loss of interest in usually enjoyed activities, problems concentrating, loneliness, self-loathing, apathy or indifference, depersonalization, loss of interest in sexual activity, shyness or social anxiety, irritability, chronic pain, lack of motivation, and morbid/suicidal ideation) to mania (e.g., elation, euphoria, irritation, and/or suspiciousness). Bipolar disorder is defined and categorized in the Diagnostic and Statistical Manual of Mental Disorders, 4^{th} Ed., Text Revision (DSM-IV-TR), American Psychiatric Assoc., 200, pages 382-401. Bipolar disorder includes bipolar I disorder, bipolar II disorder, cyclothymia, and bipolar disorder not otherwise specified.

Treatment of bipolar disorder can be assessed in clinical trials using rating scales such as the Montgomery-Asberg Depression Rating Scale, the Hamilton Depression Scale, the Raskin Depression Scale, Feighner criteria, and/or Clinical Global Impression Scale Score (Gijsman et al., Am J Psychiatry 2004, 161, 1537-1547).

### Anxiety

Anxiety is defined and categorized in the Diagnostic and Statistical Manual of Mental Disorders, 4^{th} Ed., Text Revision (DSM-IV-TR), American Psychiatric Assoc., 200, pages 429-484. Anxiety disorders include panic attack, agoraphobia, panic disorder without agoraphobia, agoraphobia without history of panic disorder, specific phobia, social phobia, obsessive-compulsive disorder, posttraumatic stress disorder, acute stress disorder, generalized anxiety disorder, anxiety disorder due to a general medical condition, substance-induced anxiety disorder, and anxiety disorder not otherwise specified. Recent work has documented a correlation of decreased levels of creatine/phosphocreatine in centrum semiovale (a representative region of the cerebral white matter) with the severity of anxiety (Coplan et al., Neuroimraging, 2006, 147, 27-39).

In clinical trials, efficacy can be evaluated using psychological procedures for inducing experimental anxiety applied to healthy volunteers and patients with anxiety disorders (see e.g., Graeff, et al., Brazilian J Medical Biological Res 2003, 36, 421-32) or by selecting patients based on the Structured Clinical interview for DSM-IV Axis I Disorders as described by First et al., Structured Clinical Interview for DSM-IV Axis I Disorders, Patient Edition (SCIDIP), Version 2. Biometrics Research, New York State Psychiatric Institute, New York, 1995. Any of a number of scales can be used to evaluate anxiety and the efficacy of treatment including, for example, the Penn State Worry Questionnaire (Behar et al., J Behav Ther Exp Psychiatr 2003, 34, 25-43), the Hamilton Anxiety and Depression Scales, the Spielberger State-Trait Anxiety Inventory, and the Liebowitz Social Anxiety Scale (Hamilton, J Clin Psychiatry 1980, 41, 21-24, Spielberger and Vagg, J Personality Assess 1984, 48, 95-97; and Liebowitz, J Clin Psychiatry, 1993, 51, 31-35 (Suppl.)).

### Genetic Diseases Affecting the Creatine Kinase System

The intracellular creatine pool is maintained by uptake of creatine from the diet and by endogenous creatine synthesis. Many tissues, especially the liver and pancreas, contain the Na⁺—Cl⁻-dependent creatine transport (SLC6A8), which is responsible for active creatine transport through the plasma membrane. Creatine biosynthesis involves the action of two enzymes: L-arginine:glycine amidinotransferase (AGAT) and guanidinoacetate transferase (GAMT). AGAT catalyses the transfer of the amidino group of arginine to glycine to generate ornithine and guanidinoacetate. Guanidino acetate is methylated at the amidino group by GAMT to give creatine (see e.g., Wyss and Kaddurah-Daouk, Phys Rev 2000, 80, 1107-213).

In humans, two genetic errors in creatine biosynthesis and one in creatine transporter are known and involve deficiencies of AGAT, GAMT, and creatine transporter (Schulze, Cell Biochem, 2003, 244(1-2), 143-50; Sykut-Cegielska et al., Acta Biochimica Polonica 2004, 51(4), 875-882). Patients with disorders of creatine synthesis have systemic depletion of creatine and creatine phosphate. Patients affected with AGAT deficiency can show mental and motor retardation, severe delay in speech development, and febrile seizures (Item et al., Am J Hum Genet. 2001, 69, 1127-1133). Patients affected with GAMT deficiency can show developmental delay with absence of active speech, autism with self-injury, extra pyramidal symptoms, and epilepsy (Stromberger et al., J Inherit Metab Dis 2003, 26, 299-308). Patients with creatine transporter deficiency exhibit intracellular depletion of creatine and creatine phosphate. The gene encoding the creatine transporter is located on the X-chromosome, and affected male patients show mild to severe mental retardation with affected females having a milder presentation (Salomons et al., J. Inherit Metab Dis 2003, 26, 309-18; Rosenberg et al., Am J Hum Genet. 2004, 75, 97-105; deGrauw et al., Neuropediatrics 2002, 33(5), 232-238; Clark et al., Hum Genet. 2006, April).

Creatine supplementation in dosages from about 350 mg to 2 g/kg body weight per day have been shown effective in resolving the clinical symptoms of AGAT or GAMT deficiencies (see e.g., Schulze, Cell Biochem. 2003, 244(1-2), 143-50). However, unlike in patients with GAMT and AGAT deficiency, in patients with creatine transporter deficiency oral creatine supplementation does not result in an increase in brain creatine levels (see Stockler-Ipsiroglu et al., in Physician's Guide to the Treatment and Follow up of Metabolic Diseases, eds Blau et al., Springer Verlag, 2004).

### Muscle Fatigue

During high-intensity exercise, ATP hydrolysis is initially buffered by creatine phosphate via the creatine kinase reaction (Kongas and van Beek, 2^{nd} Int. Conf. Systems Biol 2001, Los Angeles Calif., Omnipress, Madison, Wis., 198-207; and Walsh et al., J Physiol 2001, 537.3, 971-78, each of which is incorporated by reference herein in its entirety). During exercise, whereas creatine phosphate is available instantaneously for ATP regeneration, glycolysis is induced with a delay of a few seconds, and stimulation of mitochondrial oxidative phosphorylation is delayed even further. Because the creatine phosphate stores in muscle are limited, during high-intensity exercise, creatine phosphate is depleted within about 10 seconds. It has been proposed that muscle performance can be enhanced by increasing the muscle stores of creatine phosphate and thereby delay creatine phosphate depletion. Although creatine and/or creatine phosphate supplementation may improve muscle performance in intermittent, supramaximal exercise, there is no indication that supplementation enhances endurance performance. On the other hand, intravenous injection of creatine phosphate appears to improve exercise tolerance during prolonged submaximal exercise (Clark, J Athletic Train, 1997, 32, 45-51, which is incorporated by reference herein in its entirety).

### Muscle Strength

Dietary creatine supplementation in normal healthy individuals has beneficial side effects on muscle function, and as such its use by amateur and professional athletics has increased. There is evidence to suggest that creatine supplementation can enhance overall muscle performance by increasing the muscle store of creatine phosphate, which is the most important energy source for immediate regeneration of ATP in the first few seconds of intense exercise, by accelerating restoration of the creatine phosphate pool during recovery periods, and by depressing the degradation of adenosine nucleotides and possibly also accumulation of lactate during exercise (see e.g., Wyss and Kaddurah-Daouk, Physiol Rev 2000, 80(3), 1107-1213).

However, in normal healthy individuals, the continuous and prolonged use of creatine fails to maintain elevated creatine and creatine phosphate in muscle (see e.g., Juhn et al., Clin J Sport Med 1998, 8, 286-297; Terjung et al., Med Sci Sports Exerc 2000, 32, 706-717; and Vandenberghe et al., J Appl Physiol 1997, 83, 2055-2063, each of which is incorporated by reference herein in its entirety), possibly as a result of the down regulation of the creatine transporter activity and the transporter protein content (Snow and Murphy, Mol Cell Biochem 2001, 224(1-2), 169-181, which is incorporated by reference herein in its entirety). Thus, prodrugs of a compound of the present invention may be used to maintain, restore, and/or enhance muscle strength in a mammal, and in particular a human.

The efficacy of administering a compound of The present invention for maintaining, restoring, and/or enhancing muscle strength may be assessed using animal and human models and clinical studies. Animal models that can be used for evaluation of muscle strength are disclosed, for example, in Wirth et al., J Applied Physiol 2003, 95, 402-412 and Timson, J. Appl Physiol 1990, 69(6), 1935-1945. Muscle strength can be assessed in humans using methods disclosed, for example, in Oster, U.S. Application Publication No. 2007/0032750, Engsberg et al., U.S. Application Publication No. 2007/0012105, and/or using other methods known to those skilled in the art.

### Organ and Cell Viability

In certain embodiments, the isolation of viable brain, muscle, pancreatic or other cell types for research or cellular transplant can be enhanced by perfusing cells and/or contacting cells with an isolation or growth media containing a prodrug. In certain embodiments, the viability of a tissue, organ, or cell can be improved by contacting the tissue, organ or, cell with an effective amount of a compound of the present invention or pharmaceutical composition thereof.

### Diseases Related to Glucose Level Regulation

Administration of creatine phosphate reduces plasma glucose levels, and therefore can be useful in treating diseases related to glucose level regulation such as hyperglycemia, insulin dependent or independent diabetes, and related diseases secondary to diabetes (Kaddurah-Daouk et al., U.S. Application Publication No 2005/0256134).

The efficacy of administering a compound of the present invention for treating diseases related to glucose level regulation may be assessed using animal and human models and clinical studies. Compounds can be administered to animals such as rats, rabbits or monkeys, and plasma glucose concentrations determined at various times (see e.g., Kaddurah-Daouk and Teicher, U.S. Application Publication No. 2003/0232793). The efficacy of compounds for treating insulin dependent or independent diabetes and related diseases secondary to diabetes can be evaluated using animal models of diabetes such as disclosed, for example, in Shafrir, “Animal Models of Diabetes,” Ed., 2007, CRC Press; Mordes et al., “Animal Models of Diabetes,” 2001, Harwood Academic Press; Mathe, Diabete Metab 1995, 21(2), 106-111; and Rees and Alcolado, Diabetic Med 2005, 22, 359-370.

### Embodiments of Compositions and Routes of Administration

A compound of the present invention can be formulated as a pharmaceutical composition. In one embodiment, such a composition comprises a present compound, or a pharmaceutically acceptable salt or solvate thereof. In one embodiment, the composition further comprises a pharmaceutically acceptable carrier.

Pharmaceutical compositions can be produced using standard procedures (see, e.g., “Remington's The Science and Practice of Pharmacy,” 21st edition, Lippincott, Williams & Wilcox, 2005, incorporated herein by reference in its entirety). Pharmaceutical compositions may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds disclosed herein into preparations, which can be used pharmaceutically. Proper formulation can depend, in part, on the route of administration.

In one embodiment, the pharmaceutical compositions can provide therapeutic plasma concentrations of a compound of the present invention upon administration to a patient. In another embodiment, the compound of the present invention remains conjugated to a promoiety to form a prodrug, which during transit across the intestinal mucosal barrier provides protection from presystemic metabolism. Cleavage of the promoiety of prodrug after absorption by the gastrointestinal tract may allow the prodrug to be absorbed into the systemic circulation either by active transport, passive diffusion, or by a combination of both active and passive processes. Prodrugs can remain intact until after passage of the prodrug through a biological barrier, such as the blood-brain barrier. In certain embodiments, prodrugs provided by the present disclosure can be partially cleaved, e.g., one or more, but not all, of the promoieties can be cleaved before passage through a biological barrier or prior to being taken up by a cell, tissue, or organ. Prodrugs can remain intact in the systemic circulation and be absorbed by cells of an organ, either passively or by active transport mechanisms. In certain embodiments, a prodrug will be lipophilic and can passively translocate through cellular membranes. Following cellular uptake, the prodrug can be cleaved chemically and/or enzymatically to release the corresponding compound into the cellular cytoplasm, resulting in an increase in the intracellular concentration of the compound. In certain embodiments, a prodrug can be permeable to intracellular membranes such as the mitochondrial membrane, and thereby facilitate delivery of a prodrug, and following cleavage of the promoiety or promoieties, and the compound of the present invention, to an intracellular organelle such as mitochondria.

A pharmaceutical composition can also include one or more pharmaceutically acceptable vehicles, including excipients, adjuvants, carriers, diluents, binders, lubricants, disintegrants, colorants, stabilizers, surfactants, fillers, buffers, thickeners, emulsifiers, wetting agents, and the like. Vehicles can be selected to alter the porosity and permeability of a pharmaceutical composition, alter hydration and disintegration properties, control hydration, enhance manufacturability, etc.

The pharmaceutical composition can then be administered by any suitable routes, which include, but are not limited to administering orally, parenterally, intravenously, intraarterially, intracoronarily, pericardially, perivascularly, intramuscularly, subcutaneously, intradermally, intraperitoneally, intraarticularly, intramuscularlly, intraperitoneally, intranasally, epidurally, sublingually, intranasally, intracerebrally, intravaginally, transdermally, rectally, by inhalation spray, rectally, or topically in dosage unit formulations containing conventional nontoxic pharmaceutically acceptable carriers, adjuvants, and vehicles as desired.

In certain embodiments, a pharmaceutical composition can be formulated for oral administration. Pharmaceutical compositions formulated for oral administration can provide for uptake of a compound of the present invention throughout the gastrointestinal tract, or in a particular region or regions of the gastrointestinal tract. In certain embodiments, a pharmaceutical composition can be formulated to enhance uptake a compound of the present invention from the upper gastrointestinal tract, and in certain embodiments, from the small intestine. Such compositions can be prepared in a manner known in the pharmaceutical art and can further comprise, in addition to a compound of the present invention, one or more pharmaceutically acceptable vehicles, permeability enhancers, and/or a second therapeutic agent.

In certain embodiments, a pharmaceutical composition can further comprise substances to enhance, modulate and/or control release, bioavailability, therapeutic efficacy, therapeutic potency, stability, and the like.

Pharmaceutical compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles can be of pharmaceutical grade. For oral liquid preparations such as, for example, suspensions, elixirs, and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines, and the like may be added.

When a compound of the present invention is acidic, it may be included in any of the above-described formulations as the free acid, a pharmaceutically acceptable salt, a solvate, or a hydrate. Pharmaceutically acceptable salts substantially retain the activity of the free acid, may be prepared by reaction with bases, and tend to be more soluble in aqueous and other protic solvents than the corresponding free acid form. In some embodiments, sodium salts of a compound of the present invention are used in the above-described formulations.

The present compositions can formulated for parenteral administration including administration by injection, for example, into a vein (intravenously), an artery (intraarterially), a muscle (intramuscularly), under the skin (subcutaneously or in a depot formulation), to the pericardium, to the coronary arteries, or used as a solution for delivery to a tissue or organ, for example, use in a cardiopulmonary bypass machine or to bathe transplant tissues or organs. Injectable compositions can be pharmaceutical compositions for any route of injectable administration, including, but not limited to, intravenous, intraarterial, intracoronary, pericardial, perivascular, intramuscular, subcutaneous, intradermal, intraperitoneal, and intraarticular. In certain embodiments, an injectable pharmaceutical composition can be a pharmaceutically appropriate composition for administration directly into the heart, pericardium or coronary arteries.

The present compositions suitable for parenteral administration can comprise one or more compounds of the present invention in combination with one or more pharmaceutically acceptable sterile isotonic aqueous, water-miscible, or non-aqueous vehicles. Pharmaceutical compositions for parenteral use may include substances that increase and maintain drug solubility such as complexing agents and surface acting agents, compounds that make the solution isotonic or near physiological pH such as sodium chloride, dextrose, and glycerin, substances that enhance the chemical stability of a solution such as antioxidants, inert gases, chelating agents, and buffers, substances that enhance the chemical and physical stability, substances that minimize self aggregation or interfacial induced aggregation, substances that minimize protein interaction with interfaces, preservatives including antimicrobial agents, suspending agents, emulsifying agents, and combinations of any of the foregoing. Pharmaceutical compositions for parenteral administration can be formulated as solutions, suspensions, emulsions, liposomes, microspheres, nanosystems, and powder to be reconstituted as solutions. Parenteral preparations are described in “Remington, The Science and Practice of Pharmacy,” 21st edition, Lippincott, Williams & Wilkins, Chapter 41-42, pages 802-849, 2005.

In certain embodiments a pharmaceutical composition can be formulated for bathing transplantation tissue or organs before, during, or after transit to an intended recipient. Such compositions can be used before or during preparation of a tissue or organ for transplant. In certain embodiments, a pharmaceutical composition can be a cardioplegic solution administered during cardiac surgery. In certain embodiments, a pharmaceutical composition can be used, for example, in conjunction with a cardiopulmonary bypass machine to provide the pharmaceutical composition to the heart. Such pharmaceutical compositions can be used during the induction, maintenance, or reperfusion stages of cardiac surgery (see e.g., Chang et al., Masui 2003, 52(4), 356-62; Ibrahim et al., Eur. J. Cardiothorac Surg 1999, 15(1), 75-83; von Oppell et al., J Thorac Cardiovasc Surg. 1991, 102(3), 405-12; and Ji et al., J. Ertra Corpor Technol 2002, 34(2), 107-10). In certain embodiments, a pharmaceutical composition can be delivered via a mechanical device such as a pump or perfuser (see e.g., Hou and March, J Invasive Cardiol 2003, 15(1), 13-7; Maisch et al., Am. J Cardiol 2001, 88(11), 1323-6; and Macris and Igo, Clin Cardiol 1999, 22(1, Suppl 1), 136-9).

For prolonged delivery, a pharmaceutical composition can be provided as a depot preparation, for administration by implantation, e.g., subcutaneous, intradermal, or intramuscular injection. Thus, in certain embodiments, a pharmaceutical composition can be formulated with suitable polymeric or hydrophobic materials, e.g., as an emulsion in a pharmaceutically acceptable oil, ion exchange resins, or as a sparingly soluble derivative. e.g., as a sparingly soluble salt form of a compound of the present invention.

The present compositions can be formulated so as to provide immediate, sustained, or delayed release of a compound of the present invention after administration to the patient by employing procedures known in the art (see, e.g., Allen et al., “Ansel's Pharmaceutical Dosage Forms and Drug Delivery Systems.” 8th ed., Lippincott, Williams & Wilkins, August 2004), which is incorporated by reference in its entirety.

The present compositions can be formulated in a unit dosage form. Unit dosage form refers to a physically discrete unit suitable as a unitary dose for patients undergoing treatment, with each unit containing a predetermined quantity of a compound of the present invention calculated to produce an intended therapeutic effect. A unit dosage form can be for a single daily dose or one of multiple daily doses, e.g., 2 to 4 times per day. When multiple daily doses are used, the unit dosage can be the same or different for each dose. One or more dosage forms can comprise a dose, which may be administered to a patient at a single point in time or during a time interval.

The present compositions can be used in dosage forms that provide immediate release and/or sustained release of a compound of the present invention. The appropriate type of dosage form can depend on the disease, disorder, or condition being treated, and on the method of administration. For example, for the treatment of acute ischemic conditions such as cardiac failure or stroke the use of an immediate release pharmaceutical composition or dosage form administered parenterally may be appropriate. For treatment of chronic neurodegenerative disorders, controlled release pharmaceutical composition or dosage form administered orally may be appropriate.

In certain embodiments, a dosage form can be adapted to be administered to a patient once, twice, three times, or more frequently per day. Dosing may be provided alone or in combination with other drugs and may continue as long as required for effective treatment of the disease, disorder, or condition.

Sustained release oral dosage forms comprising a compound of the present invention can provide a concentration of the corresponding compound of the present invention in the plasma, blood, or tissue of a patient over time, following oral administration to the patient. The concentration profile of a compound of the present invention can exhibit an AUC that is proportional to the dose of the corresponding compound of the present invention.

Regardless of the specific form of controlled release oral dosage form used, a compound of the present invention can be released from an orally administered dosage form over a sufficient period of time to provide prolonged therapeutic concentrations of the compound of the present invention in the plasma and/or blood of a patient. Following oral administration, a dosage form comprising a compound of the present invention can provide a therapeutically effective concentration of the corresponding compound of the present invention in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of a compound of the present invention is maintained can be the same or different. The continuous period of time during which a therapeutically effective plasma concentration of a compound of the present invention is maintained can begin shortly after oral administration or after a time interval.

In certain embodiments, an oral dosage for treating a disease, disorder, or condition in a patient can comprise a compound of the present invention wherein the oral dosage form is adapted to provide, after a single administration of the oral dosage form to the patient, a therapeutically effective concentration of the corresponding compound of the present invention in the plasma of the patient for a first continuous time period selected from at least about 4 hours, at least about 8 hours, at least about 12 hours, and at least about 16 hours, and at least about 20 hours.

Dosage levels are dependent on the nature of the condition, drug efficacy, the condition of the patient, the judgment of the practitioner, and the frequency and mode of administration; optimization of such parameters is within the ordinary level of skill in the art. In addition, in vitro or in vivo assays may optionally be employed to help identify optimal dosage ranges. The amount of a compound administered can depend on, among other factors, the patient being treated, the weight of the patient, the health of the patient, the disease being treated, the severity of the affliction, the route of administration, the potency of the compound, and the judgment of the prescribing physician.

A compound of the present invention can be administered to an adult patient in amounts ranging from about 1-20 gram/day, 1-15 gram/day, 1-10 gram/day, or 1-5 gram/day, 3-5 gram/day, or 2-3 gram/day. For example, the present compound may be administered to an adult patient in about 20 gram/day during the initial loading phase followed by about 3 to about 5 gram/day as a maintenance dose. For a pediatric patient, the dosage amount can be about 50%, about 60, or about 70% of the one for an adult patient. For example, it may be about 12 gram/day for a pediatric patient. In certain embodiments, a therapeutically effective dose of a compound of the present invention can comprise from about 1 mg-equivalents to about 20,000 mg-equivalents, or more of a compound of the present invention per day, from about 100 mg-equivalents to about 12,000 mg-equivalents of creatine phosphate analog per day, from about 1,000 mg-equivalents to about 10,000 mg-equivalents of creatine phosphate analog per day, and in certain embodiments, from about 4,000 mg-equivalents to about 8,000 mg-equivalents of creatine phosphate analog per day.

In certain embodiments an administered dose is less than a toxic dose. Toxicity of the compositions described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition can exhibit a high therapeutic index. The data obtained from these cell culture assays and animal studies can be used in formulating a dosage range that is not toxic for use in humans. A dose of a pharmaceutical composition provided by the present disclosure can be within a range of circulating concentrations in for example the blood, plasma, or central nervous system, that include the effective dose and that exhibits little or no toxicity. A dose may vary within this range depending upon the dosage form employed and the route of administration utilized.

During treatment, a dose and dosing schedule can provide sufficient or steady state levels of an effective amount of a creatine phosphate analog to treat a disease. In certain embodiments, an escalating dose can be administered.

In one embodiment, the present invention provides a sustained release pharmaceutical composition comprising a compound of the present invention, or a pharmaceutically acceptable salt or solvate thereof, wherein the release of the compound is over a period of about 4 hours or more. In other embodiments, the release of the compound is over a period of about 5, about 6, about 7, about 8, about 9, about 10, about 11, about 12, about 13, about 14, about 15, about 16, about 17, about 18, about 19, about 20, about 21, about 22, about 23, or about 24 hours.

In another embodiment, the present invention provides a sustained release pharmaceutical composition comprising a compound of the present invention, or a pharmaceutically acceptable salt or solvate thereof, wherein the pharmacological effect from the compound lasts about 4 hours or more upon administration of the composition. In other embodiments, the pharmacological effect from the compound lasts about 5, about 6, about 7, about 8, about 9, about 10, about 11, about 12, about 13, about 14, about 15, about 16, about 17, about 18, about 19, about 20, about 21, about 22, about 23, or about 24 hours.

In another embodiment, the present invention provides a sustained release pharmaceutical composition comprising a compound of the present invention, or a pharmaceutically acceptable salt or solvate thereof; wherein the composition, upon administration, provides a therapeutically effective amount of the compound for about 4 hours or more. In other embodiments, the composition provides a therapeutically effective amount of the compound for about 5, about 6, about 7, about 8, about 9, about 10, about 11, about 12, about 13, about 14, about 15, about 16, about 17, about 18, about 19, about 20, about 21, about 22, about 23, or about 24 hours.

In one embodiment of any of the above-described sustained release pharmaceutical composition, the composition contains a matrix which comprises a compound of the present invention, or a pharmaceutically acceptable salt or solvate thereof; and one or more release rate controlling polymers. In one embodiment, the matrix is in form of a core or a layer over a core.

In one embodiment, the matrix comprises one or more polymers selected from the group consisting of a) at least one water-swellable, pH independent polymer, b) at least one anionic, pH-dependent, gel-forming copolymer, c) at least one cationic polymer, and d) at least one hydrocolloid polymer.

In one embodiment of any of the above-described sustained release pharmaceutical composition, the composition contains a release rate controlling membrane disposed over: a pull layer comprising a compound of the present invention, or a pharmaceutically acceptable salt or solvate thereof, and an osmotic push layer; wherein the release rate controlling membrane has an orifice immediately adjacent to the pull layer. In one embodiment, the pull layer further comprises a release rate controlling polymer.

In one embodiment of any of the above-described sustained release pharmaceutical composition, the composition comprise one or more particles, and each of the particles comprises an active core comprising a compound of the present invention, or a pharmaceutically acceptable salt or solvate thereof; and a release rate controlling polymer disposed over the core.

In one embodiment of any of the above-described sustained release pharmaceutical composition, the composition comprises one or more particles, and each of the particles comprises an inert core, an active layer comprising a compound of the present invention, or a pharmaceutically acceptable salt or solvate thereof disposed over the inert core, and a release rate controlling polymer disposed over the active layer.

Various sustained release systems for drugs have also been devised, and can be applied to compounds of the invention. See, for example, U.S. Pat. No. 5,624,677, International Patent Application No. PCT/US2011/043910, and U.S. patent application Ser. No. 12/595,027; the disclosures of which are incorporated herein by reference in their entireties for all purposes.

In certain embodiments, it may be desirable to introduce a compound of the present invention, a pharmaceutically acceptable salt, or a pharmaceutically acceptable solvate of any of the foregoing, or a pharmaceutical composition of any of the foregoing directly into the central nervous system by any suitable route, including intraventricular, intrathecal, and epidural injection. Intraventricular injection can be facilitated by the use of an intraventricular catheter, for example, attached to a reservoir, such as an Ommaya reservoir.

In certain embodiments, a compound of the present invention or pharmaceutical composition thereof can be administered as a single, one time dose or chronically. By chronic it is meant that the methods and compositions of the invention are practiced more than once to a given individual. For example, chronic administration can be multiple doses of a pharmaceutical composition administered to an animal, including an individual, on a daily basis, twice daily basis, or more or less frequently, as will be apparent to those of skill in the art. In another embodiment, the methods and compositions are practiced acutely. By acute it is meant that the methods and compositions of the invention are practiced in a time period close to or contemporaneous with the ischemic or occlusive event. For example, acute administration can be a single dose or multiple doses of a pharmaceutical composition administered at the onset of an ischemic or occlusive event such as acute myocardial infarction, upon the early manifestation of an ischemic or occlusive event such as, for example, a stroke, or before, during or after a surgical procedure. A time period close to or contemporaneous with an ischemic or occlusive event will vary according to the ischemic event but can be, for example, within about 30 minutes of experiencing the symptoms of a myocardial infarction, stroke, or intermittent claudication. In certain embodiments, acute administration is administration within about an hour of the ischemic event. In certain embodiments, acute administration is administration within about 2 hours, about 6 hours, about 10 hours, about 12 hours, about 15 hours or about 24 hours after an ischemic event.

In certain embodiments, a compound of the present invention or pharmaceutical composition thereof can be administered chronically. In certain embodiments, chronic administration can include several intravenous injections administered periodically during a single day. In certain embodiments, chronic administration can include one intravenous injection administered as a bolus or as a continuous infusion daily, about every other day, about every 3 to 15 days, about every 5 to 10 days, and in certain embodiments, about every 10 days.

In certain embodiments, a compound of the present invention, a pharmaceutically acceptable salt thereof, or pharmaceutically acceptable solvate of any of the foregoing, can be used in combination therapy with at least one other therapeutic agent. A compound of the present invention and other therapeutic agent(s) can act additively or, and in certain embodiments, synergistically. In some embodiments, a compound of the present invention can be administered concurrently with the administration of another therapeutic agent, such as for example, a compound for treating a disease associated with a dysfunction in energy metabolism; treating muscle fatigue; enhancing muscle strength and endurance; increasing the viability of organ transplants; and improving the viability of isolated cells. In some embodiments, a compound of the present invention, a pharmaceutically acceptable salt, or a pharmaceutically acceptable solvate of any of the foregoing can be administered prior to or subsequent to administration of another therapeutic agent, such as for example, a compound for treating a disease associated with a dysfunction in energy metabolism such as ischemia, ventricular hypertrophy, a neurodegenerative disease such as ALS, Huntington's disease, Parkinson's disease, or Alzheimer's disease, surgery related ischemic tissue damage, and reperfusion tissue damage; treating multiple sclerosis (MS), treating a psychotic disorder such as schizophrenia, bipolar disorder, or anxiety; treating muscle fatigue; enhancing muscle strength and endurance; increasing the viability of organ transplants; and improving the viability of isolated cells.

### Combinational Use

The present compositions can include, in addition to one or more compounds provided by the present disclosure, one or more therapeutic agents effective for treating the same or different disease, disorder, or condition.

Methods provided by the present disclosure include administration of one or more compounds or the present compositions and one or more other therapeutic agents provided that the combined administration does not inhibit the therapeutic efficacy of the one or more compounds provided by the present disclosure and/or does not produce adverse combination effects.

In certain embodiments, compositions provided by the present disclosure can be administered concurrently with the administration of another therapeutic agent, which can be part of the same pharmaceutical composition or dosage form as, or in a different composition or dosage form from, that containing the compounds provided by the present disclosure. In certain embodiments, compounds provided by the present disclosure can be administered prior or subsequent to administration of another therapeutic agent. In certain embodiments of combination therapy, the combination therapy comprises alternating between administering a composition provided by the present disclosure and a composition comprising another therapeutic agent, e.g., to minimize adverse side effects associated with a particular drug. When a compound provided by the present disclosure is administered concurrently with another therapeutic agent that potentially can produce adverse side effects including, but not limited to, toxicity, the therapeutic agent can advantageously be administered at a dose that falls below the threshold at which the adverse side effect is elicited.

In certain embodiments, compounds or the present compositions include, or can be administered to a patient together with, another compound for treating Parkinson's disease such as amantadine, benztropine, bromocriptine, levodopa, pergolide, pramipexole, ropinirole, selegiline, trihexyphenidyl, or a combination of any of the foregoing.

In certain embodiments, compounds or the present compositions include, or can be administered to a patient together with, another compound for treating Alzheimer's disease such as donepezil, galantamine, memantine, rivastigmine, tacrine, or a combination of any of the foregoing.

In certain embodiments, compounds or the present compositions include, or can be administered to a patient together with, another compound for treating ALS such as riluzole.

In certain embodiments, compounds or the present compositions include, or can be administered to a patient together with, another compound for treating ischemic stroke such as aspirin, nimodipine, clopidogrel, pravastatin, unfractionated heparin, eptifibatide, a β-blocker, an angiotensin-converting enzyme (ACE) inhibitor, enoxaparin, or a combination of any of the foregoing.

In certain embodiments, compounds or the present compositions include, or can be administered to a patient together with, another compound for treating ischemic cardiomyopathy or ischemic heart disease such as ACE inhibitors such as ramipril, captopril, and lisinopril; n-blockers such as acebutolol, atenolol, betaxolol, bisoprolol, carteolol, nadolol, penbutolol, propranolol, timolol, metoprolol, carvedilol, and aldosterone; diuretics; digitoxin, or a combination of any of the foregoing.

In certain embodiments, compounds or the present compositions include, or can be administered to a patient together with, another compound for treating a cardiovascular disease such as, blood-thinners, cholesterol lowering agents, anti-platelet agents, vasodilators, beta-blockers, angiotensin blockers, digitalis and is derivatives, or combinations of any of the foregoing.

In certain embodiments, compounds or the present compositions include, or can be administered to a patient together with, another compound for treating MS. Examples of drugs useful for treating MS include corticosteroids such as methylprednisolone; IFN-β such as IFN-β1a and IFN-β1b; glatiramer acetate (Copaxone®); monoclonal antibodies that bind to the very late antigen-4 (VLA-4) integrin (Tysabri®) such as natalizumab; immunomodulatory agents such as FTY 720 sphinogosie-1 phosphate modulator and COX-2 inhibitors such as BW755c, piroxicam, and phenidone; and neuroprotective treatments including inhibitors of glutamate excitotoxicity and iNOS, free-radical scavengers, and cationic channel blockers; memantine; AMPA antagonists such as topiramate; and glycine-site NMDA antagonists (Virley, NeruoRx 2005, 2(4), 638-649, and references therein; and Kozachuk, U.S. Application Publication No. 2004/0102525).

In certain embodiments, compounds or the present compositions include, or can be administered to a patient together with, another compound for treating schizophrenia. Examples of antipsychotic agents useful in treating schizophrenia include, but are not limited to, acetophenazine, alseroxylon, amitriptyline, aripiprazole, astemizole, benzquinamide, carphenazine, chlormezanone, chlorpromazine, chlorprothixene, clozapine, desipramine, droperidol, aloperidol, fluphenazine, flupenthixol, glycine, oxapine, mesoridazine, molindone, olanzapine, ondansetron, perphenazine, pimozide, prochlorperazine, procyclidine, promazine, propiomazine, quetiapine, remoxipride, reserpine, risperidone, sertindole, sulpiride, terfenadine, thiethylperzaine, thioridazine, thiothixene, trifluoperazine, triflupromazine, trimeprazine, and ziprasidone. Other antipsychotic agents useful for treating symptoms of schizophrenia include amisulpride, balaperidone, blonanserin, butaperazine, carphenazine, eplavanserin, iloperidone, lamictal, onsanetant, paliperidone, perospirone, piperacetazine, raclopride, remoxipride, sarizotan, sonepiprazole, sulpiride, ziprasidone, and zotepine; serotonin and dopamine (5HT/D2) agonists such as asenapine and bifeprunox; neurokinin 3 antagonists such as talnetant and osanetant; AMPAkines such as CX-516, galantamine, memantine, modafinil, ocaperidone, and tolcapone; and α-amino acids such as D-serine, D-alanine, D-cycloserine, and N-methylglycine.

In certain embodiments, compounds or the present compositions include, or can be administered to a patient together with, another compound for treating bipolar disorder such as aripiprazole, carbamazepine, clonazepam, clonidine, lamotrigine, quetiapine, verapamil, and ziprasidone.

In certain embodiments, compounds or the present compositions include, or can be administered to a patient together with, another compound for treating anxiety such as alprazolam, atenolol, busipirone, chlordiazepoxide, clonidine, clorazepate, diazepam, doxepin, escitalopram, halazepam, hydroxyzine, lorazepam, prochlorperazine, nadolol, oxazepam, paroxetine, prochlorperazine, trifluoperazine, and venlafaxine.

### Preparation and Examples

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons. New York, N.Y.; and Larock R. C., Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. For example, guanidine functional groups may be unstable under certain conditions and thereby need to be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

Synthesis of the examples of presented compounds, such as bis carbamates and amide guanidines as well as amides and esters, is illustrated in the following schemes and procedures.

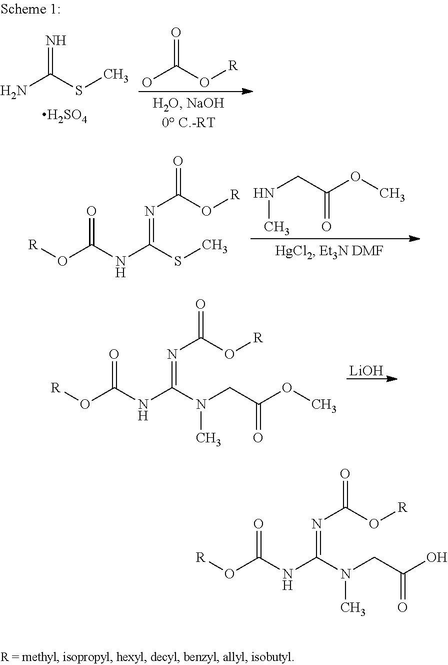

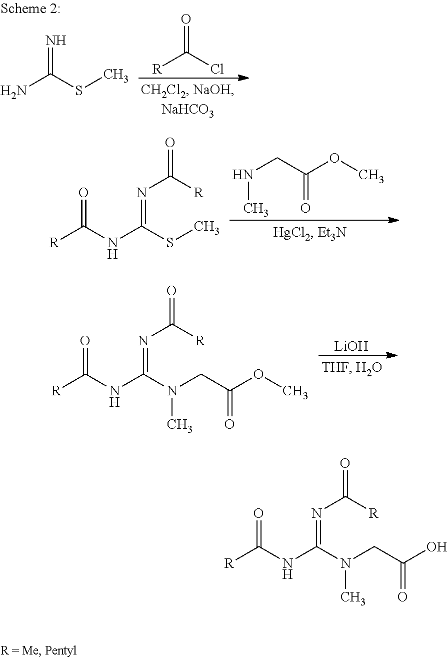

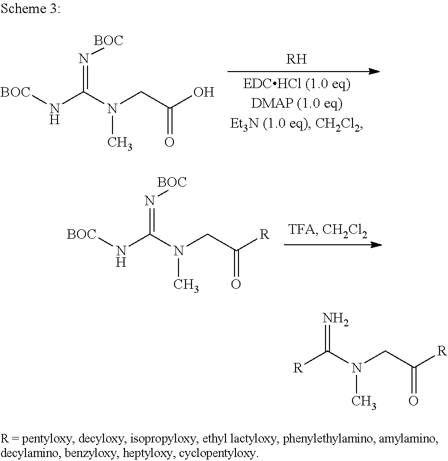

Non-limiting examples of creatine prodrugs and their PK properties are illustrated in Table 1.

TABLE 1
PK Properties

| | | | | | Polar |
|---|---|---|---|---|---|
| | | charge | | logD | Surface |
| prodrug | MW | at pH 7 | logP | at 7.4 | Area |
| creatine-serine | 219.22 | 1 | −7.86 | −4.98 | 148.92 |
| creatine-serine (−1C) | 204.18 | 0 | −1.99 | −3.64 | 148.92 |
| creatine-homoserine (+1C) | 233.25 | 1 | −7.8 | −5.73 | 148.92 |
| creatine-threonine | 233.25 | 1 | −7.44 | −4.63 | 148.92 |
| creatine-lysine | 259.3 | 1 | −7.57 | −5.59 | 151.72 |
| creatine-lysine (−1C) | 246.29 | 1 | −8.01 | −5.74 | 151.72 |
| creatine-lysine (−2C) | 232.26 | 1 | −8.53 | −6.12 | 151.72 |
| creatine-lysine (−3C) | 218.23 | 1 | −8.59 | −5.62 | 151.72 |
| creatine-tyrosine | 295.31 | 1 | −5.99 | −3.96 | 148.92 |
| creatine-tyrosine-carbamate | 422.48 | 0 | | −0.2 | 161.99 |
| creatine-carbamate | 288.28 | −1 | −2.86 | −2.17 | 131.85 |
| creatine-carbamate ring | 187.15 | 0 | −0.58 | −0.58 | 91.72 |
| creatine-pentyl ester | 202.27 | 1 | −2.05 | −1.71 | 81.15 |
| creatine-octyl ester | 244.35 | 1 | −0.72 | −0.37 | 81.15 |
| creatine-hexyloxycar- | 287.36 | 0 | 1.65 | 1.65 | 94.22 |
| bonylcarbamide alkyl ester | | | | | |
| creatine-hexyoxylcar- | 258.29 | −1 | −2.38 | −2.09 | 108.05 |
| bonylcarbamide | | | | | |
| creatine-glucose | 294.28 | 1 | −6.24 | −5.87 | 171.3 |

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A compound of Formula (I): 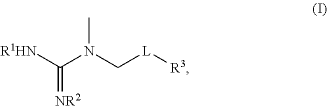
or a pharmaceutically acceptable salt or solvate thereof; wherein:
R¹ is hydrogen, —C(O)—NH—R⁴, —C(O)—O—R⁴, an amino acid residue, a dipeptide residue, or a tripeptide residue;
R² is hydrogen, —C(O)—NH—R⁵, —C(O)—O—R⁵, an amino acid residue, a dipeptide residue, or a tripeptide residue;
L is —C(O)—O— or —C(O)—NH—;
R³ is hydrogen, alkyl, alkenyl, C(O)—R⁶, an amino acid residue, a dipeptide residue, a tripeptide residue, a glucose residue, a phospholipid moiety, or a triglyceride moiety; or alternatively R¹ and R³, taken together with the atoms to which they are attached, form a heterocyclic ring;
R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
with the following provisos;
R¹, R² and R³ are not all hydrogen, but at least one of R¹, R² and R³ is hydrogen;
when R¹ and R² are hydrogen and L is —C(O)—NH—; then Formula (I) does not include a compound selected from the group consisting of Creatinyl-γ-Aminobutyric Acid Ethyl Ester, Creatinyl-L-Phenylalanine Amide, Creatinyl-L-Phenylalanine Amide, Creatinyl-Glycine Benzyl Ester, Creatinyl-Tyrosine Amide, Creatinyl-Glycine Ethylamide, Creatinyl-Phenylalanyl-Arginyl-Glycine Ethyl Ester, and Creatinyl-Phenylalanine; and
when R¹ and R² are hydrogen and L is —C(O)—O—; then R³ is not alkyl or C(O)—R⁶.

2. The compound of claim 1, when R³ is not hydrogen, then at least one of R¹ and R² is hydrogen.

3. The compound of claim 1 or 2, which demonstrates increased hydrophobicity or increased uptake by a carrier-mediated transporter as compared to the uptake of creatine, wherein the carrier-mediated transporter is selected from the group consisting of amino acid transporter, monocarboxylic acid transporter, small peptide transporter, glucose transporter, glutathione transporter, ascorbic acid transporter, and nucleoside transporter.

4. The compound of claim 1, which is represented by Formula (II): 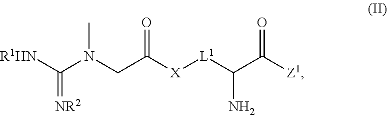
wherein:
R¹ is hydrogen, —C(O)—NH—R⁴, —C(O)—O—R⁴, an amino acid residue, a dipeptide residue, or a tripeptide residue;
R² is hydrogen, —C(O)—NH—R⁵, —C(O)—O—R⁵, an amino acid residue, a dipeptide residue, or a tripeptide residue;
X is O or NH;
L¹ is alkylene, substituted alkylene, arylene, substituted arylene; aralkylene, or substituted aralkylene;
Z¹ is C(O)—R⁶, OH, OR⁷, an amino acid residue, a dipeptide residue, a tripeptide residue, a glucose residue, a phospholipid moiety, or a triglyceride moiety;
R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
R⁷ is alkyl.

5. The compound of claim 4, wherein R¹ and R² are both hydrogen.

6. The compound of claim 5, wherein X is O or NH; and Z¹ is an amino acid residue.

7. The compound of claim 5, wherein X is O or NH; and Z¹ is a dipeptide residue or a tripeptide residue.

8. The compound of claim 4, wherein
R¹ is —C(O)—NH—R⁴, or —C(O)—O—R⁴;
R² is hydrogen;
R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl;
X is O or NH;
L¹ is alkylene, substituted alkylene, arylene, substituted arylene; aralkylene, or substituted aralkylene; and
Z¹ is OH.

9. The compound of claim 4, wherein
R¹ is hydrogen;
R² is —C(O)—NH—R⁵, or —C(O)—O—R⁵;
R⁵ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl;
X is O or NH;
L¹ is alkylene, substituted alkylene, arylene, substituted arylene; aralkylene, or substituted aralkylene; and
Z¹ is OH.

10. The compound of claim 4, wherein
R¹ is a dipeptide residue, or a tripeptide residue;
R² is hydrogen;
R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl;
X is O or NH;
L¹ is alkylene, substituted alkylene, arylene, substituted arylene; aralkylene, or substituted aralkylene; and
Z¹ is OH.

11. The compound of claim 4, wherein
one of R¹ and R² is not hydrogen;
Z¹ is OR⁷ or C(O)—R⁶;
R⁶ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
R⁷ is short-, medium, or long-chain alkyl.

12. The compound of claim 1, which is represented by Formula (III): 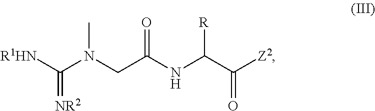
wherein:
R¹ is hydrogen, —C(O)—NH—R⁴, —C(O)—O—R⁴, an amino acid residue, a dipeptide residue, or a tripeptide residue;
R² is hydrogen, —C(O)—NH—R⁵, —C(O)—O—R⁵, an amino acid residue, a dipeptide residue, or a tripeptide residue;
Z² is OH, OR⁷, C(O)—R⁶, an amino acid residue, a dipeptide residue, a tripeptide residue, a glucose residue, a phospholipid moiety, or a triglyceride moiety; and
R, R⁴, R⁵, and R⁶ are independently alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl.

13. The compound of claim 12, wherein R¹ and R² are both hydrogen.

14. The compound of claim 13, wherein Z² is an amino acid residue.

15. The compound of claim 13, wherein Z² is a dipeptide residue or a tripeptide residue.

16. The compound of claim 12, wherein
R¹ is —C(O)—NH—R⁴, or —C(O)—O—R⁴;
R² is hydrogen;
R⁴ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
Z¹ is OH.

17. The compound of claim 12, wherein
R¹ is hydrogen;
R² is —C(O)—NH—R⁵, or —C(O)—O—R⁵;
R⁵ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
Z¹ is OH.

18. The compound of claim 12, wherein
R¹ is a dipeptide residue, or a tripeptide residue;
R² is hydrogen; and
Z¹ is OH.

19. The compound of claim 12, wherein
one of R¹ and R² is not hydrogen;
Z² is OR⁷ or C(O)—R⁶;
R⁶ is alkyl, substituted alkyl, alkenyl, substituted alkenyl, alkynyl, substituted alkynyl, heteroalkyl, substituted heteroalkyl, aryl, substituted aryl, heteroaryl, substituted heteroaryl, carbocyclyl, substituted carbocyclyl, heterocyclyl, substituted heterocyclyl; and
R⁷ is short-, medium, or long-chain alkyl.

20. The compound of claim 1, which is represented by Formula (II), wherein R¹ is —C(O)—NH—R⁴ or —C(O)—O—R⁴; R² is hydrogen and R⁴ and Z¹, taken together with the atoms to which they are attached, form a heterocyclic ring.

21. The compound of claim 1, which is represented by Formula (III), wherein R¹ is —C(O)—NH—R⁴ or —C(O)—O—R⁴; R² is hydrogen; and R⁴ and Z², taken together with the atoms to which they are attached, form a heterocyclic ring.

22. The compound of claim 1, wherein
R¹ and R² are both hydrogen;
L is —C(O)—O—; and
R³ is a glucose residue.

23. The compound of claim 1, wherein
R¹ is —C(O)—NH—R⁴ or —C(O)—O—R⁴;
R² is hydrogen;
L is —C(O)—O—;
R³ is hydrogen; and
R⁴ is heterocyclyl or substituted heterocyclyl.

24. The compound of claim 23, wherein R⁴ is a glucose residue, a nucleoside residue, or a ascorbic acid residue.

25. The compound of claim 1, wherein
R¹ and R² are both hydrogen;
L is —C(O)—O—; and
R³ is a phospholipid moiety.

26. The compound of claim 1, wherein
R¹ and R² are both hydrogen;
L is —C(O)—O—; and
R³ is a triglyceride moiety.

27. The compound of claim 1, which is selected from the group consisting of

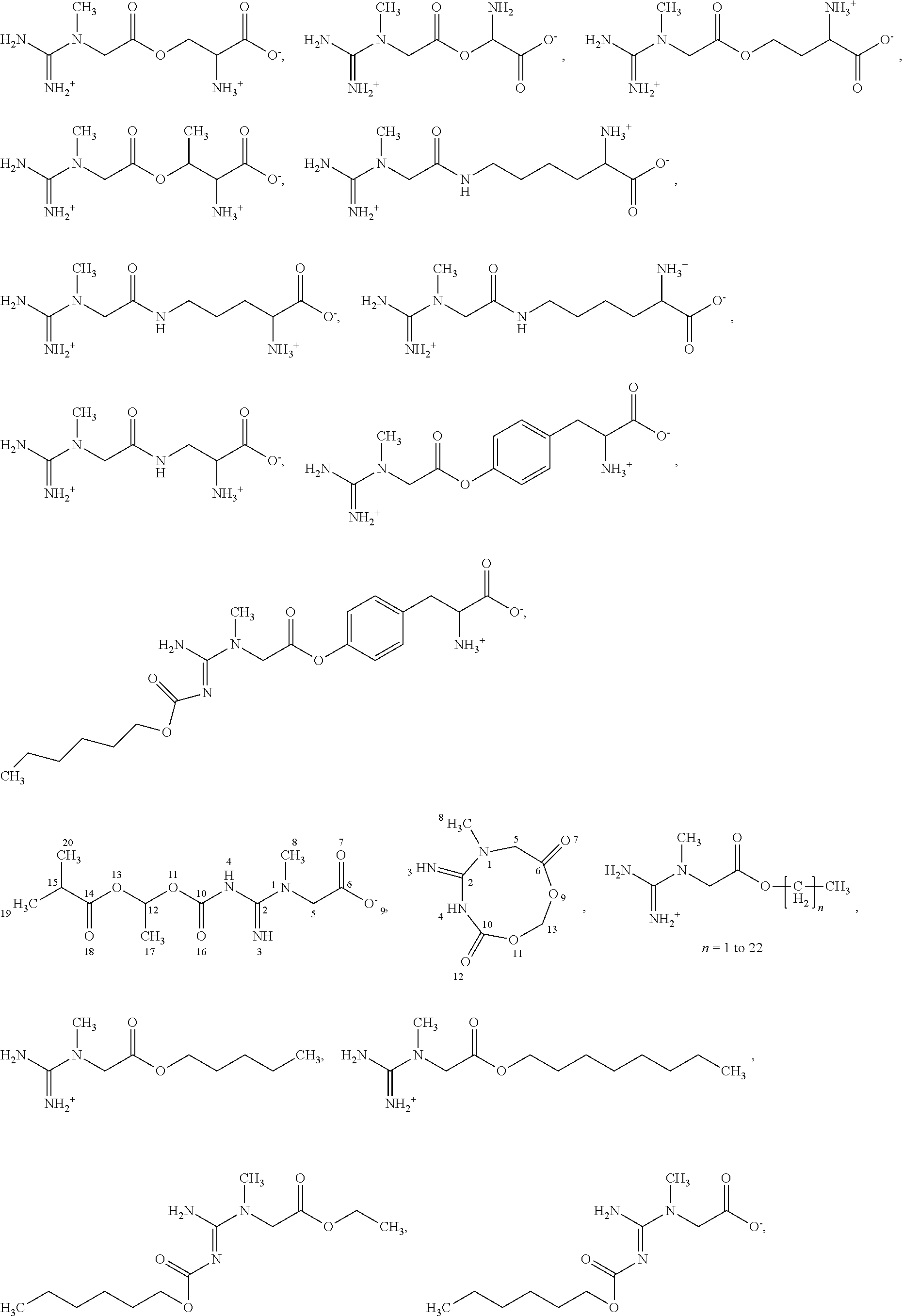

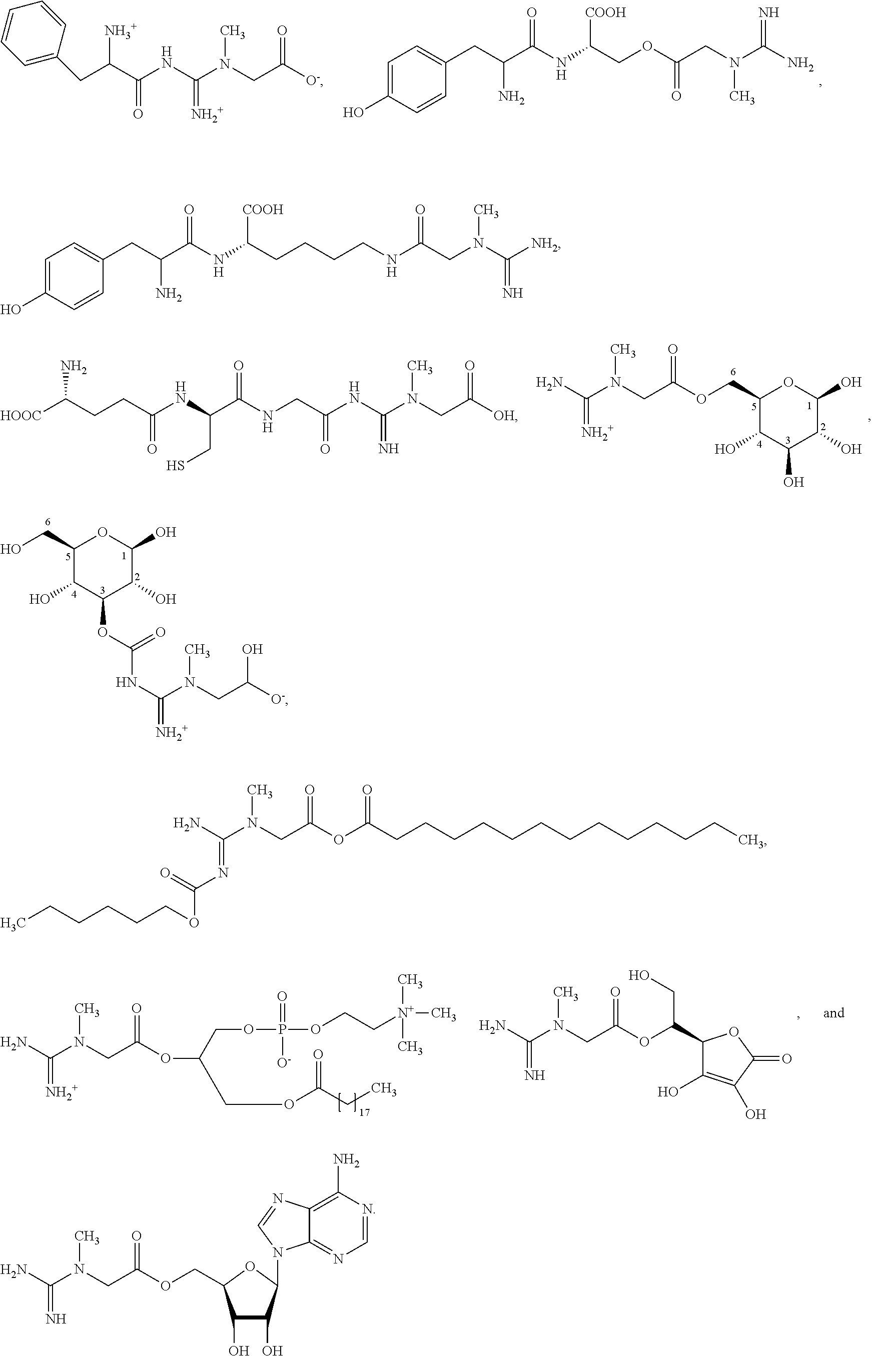

28. A pharmaceutical composition comprising a compound of any one of claims 1 to 27, or a pharmaceutically acceptable salt or solvate thereof, and a pharmaceutically acceptable carrier.

29. A method for treating creatine deficiency in a patient in need thereof comprising administering to the patient a therapeutically effective amount of a compound of any one of claims 1 to 27, or a pharmaceutically acceptable salt or solvate thereof.

30. The method of claim 29, wherein the creatine deficiency comprises a disease or condition associated with creatine transporter dysfunction.

31. The method of claim 30, wherein the disease or condition is cerebral creatine deficiency syndromes (CCDS).
